(19) United States
(12) Patent Application Publication
(10) Pub. No.: US 2014/0057314 A1
(43) Pub. Date: Feb. 27, 2014

(54) METHODS AND COMPOSITIONS FOR DETECTING MICROBIAL PRODUCTION OF WATER-IMMISCIBLE COMPOUNDS

(71) Applicant: AMYRIS, INC., Emeryville, CA (US)

(72) Inventors: Jeff Ubersax, Emeryville, CA (US); Lucas Frenz, Emeryville, CA (US)

(73) Assignee: AMYRIS, INC., Emeryville, CA (US)

(21) Appl. No.: 14/117,016

(22) PCT Filed: May 10, 2012
(86) PCT No.: PCT/US12/37351

§ 371 Date: Nov. 11, 2013

Related U.S. Application Data
(60) Provisional application No. 61/486,211, filed on May 13, 2011.

(51) Int. Cl. C12Q 1/02

(52) U.S. Cl. CPC C12Q 1/02
USPC 435/30

(57) ABSTRACT

Provided herein are methods and compositions useful for detecting the production of compounds in a cell, for example, a microbial cell genetically modified to produce one or more such compounds at greater yield and/or with increased persistence compared to a parent microbial cell that is not genetically modified. In some embodiments, the methods comprise contacting a solution with a fluorescent dye that directly binds the recombinantly produced compound, wherein the solution comprises a plurality of cells recombinantly producing the compound; and detecting the fluorescent dye under spectral conditions suitable for the selective detection of the fluorescent dye bound to the recombinantly produced compound.

### 1. CROSS-REFERENCE OF RELATED APPLICATIONS

This application is the National Stage of International Patent Application No. PCT/US2012/037351, filed May 10, 2012, which claims priority to U.S. Provisional Patent Application No. 61/486,211, filed May 13, 2011, each of which is hereby incorporated by reference in its entirety.

### 2. FIELD OF THE INVENTION

The methods and compositions provided herein generally relate to the industrial use of microorganisms. In particular, provided herein are methods and compositions useful for detecting the production of an industrially useful compound in a cell, for example, a microbial cell genetically modified to produce one or more such compounds at greater yield and/or with increased persistence compared to a parent microbial cell that is not genetically modified.

### 3. BACKGROUND

The utilization of microbes for the production of commercially useful compounds has led to the emergence of industrial biotechnology. Commercially productive strains can be derived through metabolic engineering, that is, the directed modification of metabolic fluxes in a host cell. A particular aim of metabolic engineering is to increase the intracellular concentration or secretion of valuable compounds while making other fluxes optimal for viability and productivity. Particularly desirable are recombinant strains capable of high yield (grams of compound per gram of substrate), high production (grams per liter) and/or high productivity (grams per liter per hour). Engineering of a strain to a desired phenotype is often carried out as an iterative process involving several rounds of engineering, analysis and modeling of metabolic fluxes. However, all methods of metabolic engineering share a common limitation, that is, their dependence on suitable screening methods for the improved trait.

Screening methods for strains having improved performance are ideally: (1) sensitive enough to discern incremental improvements in performance from one modified population to the next; (2) specific enough to distinguish endogenous molecules from recombinantly produced heterologous products, whether intracellularly contained or secreted into surrounding media; (3) robust enough to screen many libraries of modified strains at once; and (4) informative as to the metabolic impact of production on the host. With regard to the latter, it can be the case that the importation and expression of heterologous genes in the host will lead to metabolic imbalance and/or the accumulation of toxic metabolites. In such scenarios, it is useful to know whether production comes at the cost of reduced viability of the host. Furthermore, in view of the possibilities for widely divergent biomass from one producing population to the next, the ability to detect recombinant product specifically and without influence or input from cell biomass can provide a more accurate depiction of the yield, production, and/or productivity of a given strain.

High throughput screening methods such as robotic microtiter plate assays or fluorescence-associated cell sorting have been previously described. However, with the development of new applications for metabolic engineering, more sensitive, product-specific, and robust assays are needed, particularly those which provide some indication of the compatibility between production of the desired compound and host cell viability.

### 4. SUMMARY OF THE INVENTION

Provided herein are methods and compositions useful for detecting a water-immiscible compound (WIC) recombinantly produced in a cell, for example, a microbial cell genetically modified to produce one or more water-immiscible compounds at greater yield and/or with increased persistence compared to a parent microbial cell that is not genetically modified. In particular, the methods provided herein provide for high-throughput, sensitive and quantitative means for screening microbial strains that are engineered, for example, to produce industrially useful water-immiscible compounds, including but not limited to isoprenoids, polyketides, fatty acids, and derivatives thereof. The methods allow for the specific detection of heterologous intracellular or secreted compounds through the use of a fluorescent dye capable of directly binding the water immiscible compound, and selected spectral conditions which enable the interrogation of a recombinant cell population for the amount of compound produced relative to its biomass.

In a first aspect, provided herein is a method of detecting, in a solution, water-immiscible compound (WIC) recombinantly produced from a plurality of cells, the method comprising:

(a) contacting the solution with a fluorescent dye that directly binds the WIC, wherein the solution comprises a plurality of cells recombinantly producing the WIC; and

(b) detecting the fluorescent dye under spectral conditions suitable for the selective detection of the fluorescent dye bound to the recombinantly produced WIC.

In some embodiments, the WIC is secreted from said cells recombinantly producing said WIC. In some embodiments, the fluorescent dye is Nile Red. In some embodiments, the fluorescent dye is BODIPY 493/503 or BODIPY 505/515.

In some embodiments, the solution comprising the plurality of cells is contained in a well of a multi-well cell culture plate. In some embodiments, the cells are cultured for a period of at least 12 hours prior to said detecting.

In some embodiments, the methods further comprise the step of determining a WIC:cell biomass ratio. In some embodiments, the cell biomass is determined by a method comprising detecting the autofluorescence of said plurality of cells using spectral conditions that do not detect fluorescence from the fluorescent dye bound to the WIC. In some embodiments, the fluorescent dye is Nile Red, and determining the WIC:cell biomass ratio comprises determining the ratio of green to red fluorescence.

In some embodiments, the spectral conditions suitable for specifically detecting WIC are determined by a method comprising:

(a) contacting the fluorescent dye with a first plurality of cell populations and a second plurality of cell populations, wherein cells of the first and second plurality are of the same cell type as the WIC-producing cells to be screened, wherein each plurality comprises a cell population having a cell density of x and a cell population having a cell density of 5x, wherein each of the cell populations of the first plurality comprise WIC, and the cell populations of the second plurality do not comprise WIC;

(b) determining an excitation spectrum for the first plurality and the second plurality, respectively; and

(c) selecting an excitation wavelength wherein:
(i) the difference in fluorescence between a cell population from the first plurality and a cell population from the second plurality having the same cell density is at least 80%; and
(ii) the difference in fluorescence between cell populations having cell density x and cell density 5x from the second plurality is no greater than 250%.

In some embodiments, the emission wavelength of the excitation spectrum of step (b) is fixed at 550 nm.

In some embodiments, the spectral conditions suitable for specifically detecting WIC are determined by a method comprising:

(a) contacting the fluorescent dye with a first plurality of cell populations and a second plurality of cell populations, wherein cells of the first and second plurality are of the same cell type as the WIC-producing cells to be screened, wherein each plurality comprises a cell population having a cell density of x and a cell population having a cell density of 5x, wherein each of the cell populations of the first plurality comprise WIC, and the cell populations of the second plurality do not comprise WIC;

(b) determining an emission spectrum for the first plurality and the second plurality, respectively; and

(c) selecting an emission wavelength wherein:
(i) the difference in fluorescence between a cell population from the first plurality and a cell population from the second plurality having the same cell density is at least 80%; and
(ii) the difference in fluorescence between cell populations having cell density x and cell density 5x from the second plurality is no greater than 250%.

In some embodiments, the excitation wavelength of the emission spectrum of step (b) is fixed at 290 nm.

In some embodiments, the cell populations of the first plurality comprise at least 2 g/L of the WIC.

In some embodiments, the recombinantly produced water-immiscible compound is an isoprenoid. In some embodiments, the recombinantly produced water-immiscible compound is a terpene, C₅ isoprenoid, C₁₀ isoprenoid or C₁₅ isoprenoid. In some embodiments, the recombinantly produced water-immiscible compound is farnesene.

In another aspect, provided herein is a method of detecting, in solution, farnesene produced and secreted from a cell, the method comprising:

(a) contacting a solution with Nile Red, wherein the solution comprises a cell recombinantly producing and secreting farnesene; and

(b) detecting Nile Red at an excitation wavelength of about 260 to 290 nm and an emission wavelength of about 530 to 570 nm.

In some embodiments, the cell is selected from the group consisting of a yeast cell, a bacterial cell, a mammalian cell, a fungal cell, an insect cell, and a plant cell. In some embodiments, the cell is a yeast cell. In some embodiments, the yeast is Saccharomyces cerevisiae.

In another aspect, provided herein is a liquid composition comprising:

(a) a cell recombinantly producing and secreting a water-immiscible compound;

(b) water immiscible-compound secreted from said cell;

(c) a fluorescent dye that directly binds to the secreted water-immiscible compound; and

(d) cell culture medium.

### 5. BRIEF DESCRIPTION OF THE FIGURES

FIG. 1 provides a cell/farnesene titration matrix stained with Nile Red, and detected at an excitation wavelength of 488 nm and an emission wavelength of 515 nm. Populations of naïve yeast cells of OD 5, 10, 15, 20 and 25, and a no-cell control were plated in growth medium along the x-axis of a 96-well microtiter plate, while increasing concentrations of purified farnesene (0, 2, 4, 6, 8 and 10 g/L) were added to wells along the y-axis.

FIG. 2 provides a cell/farnesene titration matrix stained with Nile Red, and detected at an excitation wavelength of 500 nm and an emission wavelength of 550 nm. (A) Populations of naïve yeast cells of OD 5, 10, 15, 20 and 25, and a no-cell control were plated in growth medium along the x-axis of a 96-well microtiter plate, while increasing concentrations of purified farnesene (0, 2, 4, 6, 8 and 10 g/L) were added to wells along the y-axis. (B) A plot of farnesene concentration versus fluorescence units across increasing cell density at 500ₑₓ/550ₑₘ. R²=0.650.

FIG. 3A provides an excitation spectra from 250 to 520 nm at an emission wavelength of 550 nm. (⋄) 10 g/L farnesene, without cells; (□) naïve yeast cells of OD 25, without farnesene; and (Δ) 10 g/L farnesene plus nave yeast cells of OD 25.

FIG. 3B provides an emission spectra from 330 to 710 nm at an excitation wavelength of 290 nm. (⋄) 10 g/L farnesene, without cells; (□) naïve yeast cells of OD 25, without farnesene; and (Δ) 10 g/L farnesene plus naïve yeast cells of OD 25.

FIG. 4 provides a cell/farnesene titration matrix stained with Nile Red, and detected at an excitation wavelength of 290 nm and an emission wavelength of 550 nm. (A) Populations of naïve yeast cells of OD 5, 10, 15, 20 and 25, and a no-cell control were plated in growth medium along the x-axis of a 96-well microtiter plate, while increasing concentrations of purified farnesene (0, 2, 4, 6, 8 and 10 g/L) were added to wells along the y-axis. (B) A plot of farnesene concentration versus fluorescence units across increasing cell density at 290ₑₓ/550ₑₘ. R²=0.918.

FIG. 5 depicts the emission spectra from 430 nm to 750 nm at an excitation wavelength of 350 nm. (⋄) 10 g/L farnesene, without cells; (□) naïve yeast cells of OD 25, without farnesene; and (Δ) 10 g/L farnesene plus naïve yeast cells of OD 25.

FIG. 6 provides a cell/farnesene titration matrix stained with Nile Red, and detected at an excitation wavelength of 350 nm and an emission wavelength of 490 nm. (A) Populations of naïve yeast cells of OD 5, 10, 15, 20 and 25, and a no-cell control were plated in growth medium along the x-axis of a 96-well microtiter plate, while increasing concentrations of purified farnesene (0, 2, 4, 6, 8 and 10 g/L) were added to wells along the y-axis. (B) A plot of cell density versus fluorescence units across increasing farnesene concentration at 350ₑₓ/490ₑₘ. R²=0.955.

### 6. DETAILED DESCRIPTION OF THE EMBODIMENTS

### 6.1 Definitions

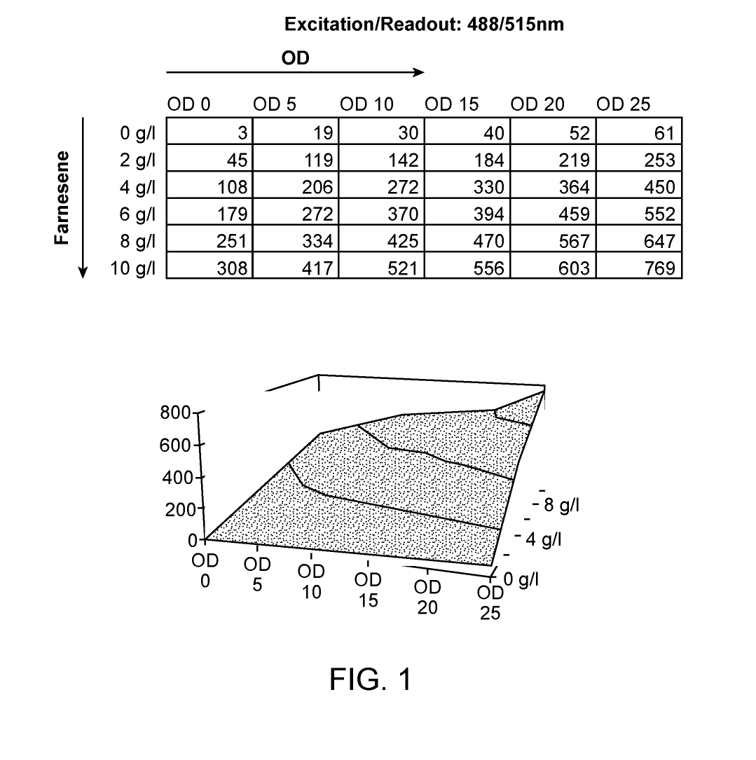

As used herein, the term “mevalonate pathway” or “MEV pathway” is used herein to refer to the biosynthetic pathway that converts acetyl-CoA to IPP. The MEV pathway is illustrated schematically in FIG. 1A.

As used herein, the term “deoxyxylulose 5-phosphate pathway” or “DXP pathway” is used herein to refer to the pathway that converts glyceraldehyde-3-phosphate and pyruvate to IPP and DMAPP. The DXP pathway is illustrated schematically in FIG. 1B.

As used herein, the phrase “heterologous nucleotide sequence” refers to a nucleotide sequence which may be: (a) foreign to its host cell (i.e., is “exogenous” to the cell); (b) naturally found in the host cell (i.e., “endogenous”) but present at an unnatural quantity in the cell (i.e., greater or lesser quantity than naturally found in the host cell); or (c) be naturally found in the host cell but positioned outside of its natural locus.

As used herein, the term “persistent” in the context of production of an isoprenoid by a genetically modified microbial cell refers to the ability of the genetically modified microbial cell to produce an isoprenoid compound over longer time spans in an industrial fermentation, compared to a non-genetically modified parent microbial cell.

As used herein, the term “parent” refers to a cell that serves as a starting point for introduction of genetic modifications that leads to the generation of a genetically modified microbial cell as described herein, e.g., genetically modified to effect increased production and/or increased levels of a water-immiscible compound, e.g., an isoprenoid, a polyketide or a fatty acid, within the cell, but does not comprise all of the genetic modifications of the genetically modified cell.

As used herein, the phrases “recombinantly produced water-immiscible compound”, “heterologous water-immiscible compound” and “WIC” refer to a compound produced from a genetically modified cell or microorganism having at least four carbon atoms wherein the compound is immiscible with water. The compound having at least four carbon atoms may be branched, linear or cyclic and optionally can include one or more heteroatoms (e.g., nitrogen, oxygen and sulfur) as well as one or more substituents or functional moieties (e.g., —OH, —NH2, —COOH, —C(H)═O, —NO3, —NH—, —C(═O)—, and the like). In some embodiments, the compound is an oil. In other embodiments, the compound is hydrophobic. Exemplary recombinantly produced, i.e. heterologous water-immiscible compounds of the methods and compositions provided herein include, but are not limited to, isoprenoids, polyketides, and fatty acids. In some embodiments, the recombinantly produced, i.e. heterologous water-immiscible compound comprises a carbon chain ranging in length from 4 carbon atoms to 40 carbon atoms. In some embodiments, the recombinantly produced, i.e. heterologous water-immiscible compound comprises a carbon chain of 5 to 30, 10 to 25, or 15 to 20 carbon atoms. In some embodiments, the recombinantly produced, i.e. heterologous water-immiscible compound comprises a carbon chain of greater than 5, 10, 15 or 20 carbon atoms. In some embodiments, the recombinantly produced, i.e. heterologous water-immiscible compound comprises a carbon chain of less than 40 carbon atoms.

As used herein, the phrase “selectively detect” or “selectively detecting” refers to the detection of a fluorescent species in a sample under select spectral conditions that largely eliminate fluorescence from other molecular species in the sample. In some embodiments, a fluorescent dye bound to a plurality of molecular species in a cell can be subjected to specific excitation/emission wavelengths such that only a subset of the species bound by the dye are detected.

As used herein, the phrase “spectral conditions” refers to optical parameters including but not limited to an excitation wavelength, an emission wavelength, and an excitation/emission wavelength pairing. The excitation wavelength is the wavelength of the radiation used to stimulate fluorescence in a sample, e.g., a solution comprising a florescent dye bound to a WIC. The emission wavelength is the wavelength of the radiation emitted by the sample being measured, e.g., the fluorescent dye.

### 6.2 Methods for Detecting Recombinantly Produced Water-Immiscible Compound

In a first aspect, provided herein is a method of detecting, in solution, water-immiscible compound (WIC) recombinantly produced from a plurality of cells, the method comprising:

(a) contacting a solution with a fluorescent dye that directly binds the WIC, wherein the solution comprises a plurality of cells recombinantly producing the WIC; and

(b) detecting the fluorescent dye under spectral conditions suitable for the selective detection of the fluorescent dye bound to the recombinantly produced WIC.

6.2.1 Contacting WIC-Producing Cells in Solution

In some embodiments, WIC may be contacted with the fluorescent dye in solution comprising cells recombinantly producing the WIC, for example, contained in a culture vessel, such as a cell culture vessel. The culture vessel can be any vessel including, without limitation, culture dishes or a well of a multiwell plate, e.g., a 96-well plate to be used specifically for performing the detection assay. In some embodiments, the vessel is made from polystyrene, polytetrafluoroethylene (PTFE), polypropylene, polycarbonate, polyvinylchloride, or other similar solid polymeric substrate. In particular embodiments, the solution comprising cell recombinantly producing the WIC is contained in a black 96-well polystyrene flat bottom assay plate.

In some embodiments, the solution comprises suitable media for culturing microbial cells producing the WIC. In some embodiments, the carbon source is a monosaccharide (simple sugar), a disaccharide, a polysaccharide, a non-fermentable carbon source, or one or more combinations thereof. Non-limiting examples of suitable monosaccharides include glucose, galactose, mannose, fructose, ribose, and combinations thereof. Non-limiting examples of suitable disaccharides include sucrose, lactose, maltose, trehalose, cellobiose, and combinations thereof. Non-limiting examples of suitable polysaccharides include starch, glycogen, cellulose, chitin, and combinations thereof. Non-limited examples of suitable non-fermentable carbon sources include acetate and glycerol. In some embodiments, the suitable medium is supplemented with one or more additional agents, such as, for example, an inducer (e.g., when one or more nucleotide sequences encoding a gene product is under the control of an inducible promoter), a repressor (e.g., when one or more nucleotide sequences encoding a gene product are under the control of a repressible promoter), or a selection agent (e.g., an antibiotic to select for microbial cells comprising the genetic modifications).

In some embodiments, the cells are cultured under conditions suitable for heterologous water-immiscible compound production. In some embodiments, the cells are cultured for a period of at least 12 hours, for a period of 12 to 24 hours, for a period of at least 24 hours, or for a period of about 36, 48, 60, 72, 96 or more than 96 hours prior to contact with the fluorescent dye. In some embodiments useful for high-throughput applications, the cells are grown in 96-well plates, and the plate is sealed with a breathable membrane seal for the duration of the culture period to prevent volume loss due to evaporation, and to allow adequate oxygen transfer to maintain an aerobic culture. In other embodiments where multiple plates of cells are stacked in an incubator, the plates are separated by 1 cm rubber gaskets to minimize positional bias. In particular embodiments, the cells are shaken during the entirety of the culture period. In some embodiments, the cells are shaken at 1000 RPM.

In some embodiments, the solution comprising the cells recombinantly producing the WIC is contacted with the fluorescent dye with no prior processing of the cells, e.g., without chemical or thermal permeabilization of the cells that may enhance uptake of the fluorescent dye. In other embodiments, the cells are treated to enhance uptake of the dye, for example, by contacting the cells with DMSO or subjecting the cells to heat treatment prior to contact with the dye.

In some embodiments, the method comprises contacting the solution comprising the cells with a fluorescent dye that directly binds to the recombinantly produced water-immiscible compound and detecting the fluorescent dye within the solution. In some embodiments, the fluorescent dye is a solvatochromic dye. Fluorescent solvatochromic dyes are dyes that change color depending on the polarity of the solvent surrounding the molecules and are used, for example, as probes in high sensitivity real time observations of dynamics of biological molecules, particularly of lipid molecules. The color changing mechanism thereof is achieved through direct binding and does not require contact with specific chemical species. Such fluorescent solvatochromic dyes include NBD, Dansyl, DASPMI, Prodan, Dapoxyl, 4-DMAP, 4-amino-1,8-naphthalimide derivatives, Reichardt's dye, and Nile Red.

In some embodiments, the solution is contacted with a BODIPY fluorophore derivative. BODIPY fluorophore derivatives feature a nonpolar structure and long-wavelength absorption and fluorescence, small fluorescence Stokes shifts, extinction coefficients that are typically greater than 80,000 cm⁻¹ M⁻¹ and high fluorescence quantum yields that are not diminished in water. BODIPY dyes have potential applications as stains for neutral lipids and as tracers for oils and other nonpolar liquids. Staining with the BODIPY 493/503 dye has been shown by flow cytometry to be more specific for cellular lipid droplets than staining with Nile Red. Moreover, the low molecular weight of the BODIPY 493/503 dye (262 Daltons) results in the probe having a relatively fast diffusion rate in membranes. The BODIPY 493/503 dye has also been used to detect neutral compounds in a microchip channel separation device. BODIPY 505/515 has been reported to permeate cell membranes of live zebrafish embryos, selectively staining cytoplasmic yolk platelets.

In some embodiments, the solution is contacted with the fluorescent dye Nile Red. Nile Red is a lipid-soluble fluorescent dye that has frequently been used for the detection of intracellular lipid droplets by fluorescence microscopy and flow cytofluorometry, for example, to evaluate the lipid content of animal cells and microorganisms, including mammalian cells, bacteria, yeasts and microalgae. Nile Red has several unique properties that make it ideal for the high throughput detection of recombinantly produced water-immiscible compounds described herein. For example, Nile Red is highly fluorescent in a hydrophobic environment, is quenched in a hydrophilic environment, and exhibits solvatochromism, that is, its excitation and emission spectra vary in spectral position, shape, and intensity with the nature of its environment. The solvatochromic property of Nile Red allows for the partial differentiation of Nile Red bound to phospho- and polar lipids and that bound to neutral lipids. In a polar lipid, such as the phospholipid cell membrane, Nile Red has a fluorescence emission maximum of ˜590 nm. By contrast, in the presence of a neutral lipid, for example, a hydrocarbon product (e.g., farnesene), the spectrum is blue-shifted with an emission maximum of 550 nm. Thus, in certain embodiments of the methods described herein, optical filters in the green (525+/−20 nm) and red (670+/−20 nm) regions of the spectrum are used during detection in order to maximize the ratio of green to red fluorescence between the ideal producing cell (e.g., pure farnesene) and a complete non-producing cell. Fluorescence data can be captured in both the green and red spectrums, and the ratio of green to red fluorescence can be used to determine the amount of water-immiscible compound within the solution normalized to the amount of cell biomass in the solution. Thus, the methods provided herein advantageously utilize solvatochromic dyes such as Nile Red to simultaneously determine: (a) the amount of water-immiscible compound produced by a cell population; and (b) the cell biomass of the population. By obviating the requirement for separate determinations of cell biomass, for example, by counterstaining the cell population with a cell wall or nuclear specific stain, or measuring the optical density of the cell population, higher throughput and efficiency can be achieved compared to other screening methods.

The ratio of green to red fluorescence (G/R) of a cell population contained in solution in a culture vessel can be advantageously used to determine the relative product:biomass ratios of the cell population, and the population can be ranked accordingly. For example, a cell population can be ranked as having: (a) a relatively high G/R ratio, which may indicate a relatively slow growing/high producing population; or (b) a relatively low G/R ratio, which may indicate a relatively fast growing/low producing population, a relatively fast growing/high producing population, or a relatively slow growing/low producing strain. The G/R ratio of the cell population can further be used in combination with its green fluorescence value alone (G), which is indicative of the amount of compound produced by the population, to further characterize the population. For example, a cell population having a low G/R ratio but high G value may indicate a relatively fast growing/high producing population, and a cell population having a low G/R ratio but low G value may indicate a relatively slow growing/low producing population or fast growing/low producing population.

Thus, in some embodiments of the methods of detecting provided herein, the method comprises normalizing the amount of water-immiscible compound of a cell population in solution within a culture vessel to the amount of cell biomass within the culture vessel. In some embodiments, said normalizing comprises determining: (a) the level of fluorescence of the water immiscible compound within the culture vessel, and (b) the level of fluorescence of cell biomass within the culture vessel; and determining the ratio of fluorescence determined in (a) to that determined in (b). In some embodiments, the fluorescent dye is Nile Red, and said normalizing comprises determining the level of fluorescence within the green spectrum (e.g., 525+/−20 nm), corresponding to the level of water-immiscible compound within the culture vessel, and determining the level of fluorescence within the red spectrum (670+/−20 nm), corresponding to the level of cell biomass within the culture vessel, and determining the ratio of green to red fluorescence (G/R). In some embodiments, the methods further comprise selecting a cell population having a high G/R ratio. In some embodiments, the methods further comprise selecting a cell population having a high level of green fluorescence. In some embodiments, the methods further comprise selecting a cell population having a high G/R ratio and a high level of green fluorescence.

6.2.2 Detection

Recombinantly produced water-immiscible compound produced from a cell or clonal population of cells can be detected using standard cell detection techniques such as flow cytometry, cell sorting, fluorescence activated cell sorting (FACS), magnetic activated cell sorting (MACS), or by light or confocal microscopy. In particular embodiments, fluorescence from water-immiscible compound producing cells is quantified in a 96-well plate fluorescence spectrophotometer.

6.2.2.1 Selecting Spectral Conditions for Detection

The determination of spectral conditions suitable for the selective detection of fluorescent dye bound to WIC produced from a plurality of cells can be carried out in several embodiments. In one embodiment, for any combination of: (1) WIC recombinantly produced by a plurality of cells; (2) a fluorescent dye that directly binds the WIC; and (3) a host cell, the spectral conditions can be determined by a method comprising the step of identifying an excitation wavelength that enables the specific detection of the dye bound to the WIC. In some embodiments, the method comprises the step of identifying an emission wavelength that enables the specific detection of the dye bound to the WIC. In some embodiments, the method comprises the step of identifying an excitation and emission wavelength pairing that enables the specific detection of the dye bound to the WIC. In preferred embodiments, the method comprises identifying an excitation and emission wavelength pairing that is sufficiently selective for the detection of fluorescent dye bound to the WIC, such that fluorescence from the host cell biomass is not detected.

In some embodiments, the method of determining spectral conditions selective for detecting fluorescent dye bound to WIC comprises determining a compatible excitation wavelength. In one embodiment, a compatible excitation wavelength is determined by:

(a) contacting the fluorescent dye with a first plurality of cell populations and a second plurality of cell populations, wherein cells of the first and second plurality are of the same cell type as the WIC-producing cells to be screened, wherein each plurality comprises a cell population having a cell density of x and a cell population having a cell density of 5x, wherein each of the cell populations of the first plurality comprise WIC, and the cell populations of the second plurality do not comprise WIC;

(b) determining an excitation spectrum for the first plurality and the second plurality, respectively; and

(c) selecting an excitation wavelength wherein:
(i) the difference in fluorescence between a cell population from the first plurality and a cell population from the second plurality having the same cell density is at least 80%; and
(ii) the difference in fluorescence between cell populations having cell density x and cell density 5x from the second plurality is no greater than 250%.

In some embodiments, the method of determining spectral conditions sufficient to selectively detect fluorescent dye bound to WIC comprises determining a compatible emission wavelength. In one embodiment, a compatible emission wavelength is determined by:

(a) contacting the fluorescent dye with a first plurality of cell populations and a second plurality of cell populations, wherein cells of the first and second plurality are of the same cell type as the WIC-producing cells to be screened, wherein each plurality comprises a cell population having a cell density of x and a cell population having a cell density of 5x, wherein each of the cell populations of the first plurality comprise WIC, and the cell populations of the second plurality do not comprise WIC;

(b) determining an emission spectrum for the first plurality and the second plurality, respectively; and

(c) selecting an emission wavelength wherein:
(i) the difference in fluorescence between a cell population from the first plurality and a cell population from the second plurality having the same cell density is at least 80%; and
(ii) the difference in fluorescence between cell populations having cell density x and cell density 5x from the second plurality is no greater than 250%.

In particular embodiments, the method of determining spectral conditions sufficient to selectively detect fluorescent dye bound to WIC comprises selecting both an excitation and emission wavelength, i.e., a compatible emission and excitation wavelength pairing, wherein (i) the difference in fluorescence between a cell population from the first plurality and a cell population from the second plurality having the same optical density is at least 80%; and (ii) the difference in fluorescence between cell populations from the second plurality of OD5 and OD25 is no greater than 250%.

Where the method comprises determining an excitation spectrum for the first and second plurality of cells, the emission wavelength is held constant, and an excitation spectrum is obtained, for example, from 250 nm to 500, or a subset of wavelengths thereof. In some embodiments, the emission wavelength is held constant at a wavelength just outside the range of excitation wavelengths of the excitation spectrum being obtained. In particular embodiments, the emission wavelength is held constant at 550 nm. Similarly, where the method comprises determining an emission spectrum for the first and second plurality of cells, the excitation wavelength is held constant, and an emission spectrum is obtained, for example, from 260 nm to 720, or a subset of wavelengths thereof. In particular embodiments, the excitation wavelength is held constant at 290 nm. Any fluorometer known in the art capable of obtaining fluorescence spectra may be used in the methods described herein.

The first and second pluralities of cell populations useful in the methods described above are preferably contained within a liquid medium that does not contribute an appreciable amount of background fluorescence to the assay. For example, the cells may be added to a well of a microtiter plate in an aqueous solution commonly used in cell culture or cell-based assays, for example, biological buffers, e.g., phosphate buffered saline, or any medium that can support the growth of cells.

In some embodiments, the cell density x of a cell population is the optical density of the cell population at 600 nm (OD₆₀₀). For example, where a first cell population having a cell density x has an OD₆₀₀ of 1, a cell population having a cell density 5x has an OD₆₀₀ of 5. In some embodiments, the first and second pluralities of cells each comprise at least two cell populations of increasing cell density, for example, cell populations of x and 5x (e.g., OD₆₀₀ of 1 and 5), x and 10x (e.g., OD₆₀₀ of 1 and 10), or x and 20x (e.g., OD₆₀₀ of 1 and 20). In some embodiments, the first and second pluralities comprise populations of lower or higher optical densities. For example, the first and second pluralities may further comprise cell populations of OD 1, 2, 3, 4, 6, 7, 8, 9, 10, 11, 12, 13, 14, 15, 16, 17, 18, 19, 20, 21, 22, 23, 24, 30, 35, 40, 45, or higher than 50. In some embodiments, the first and second pluralities comprise at least 3, 4, 5, 6, 7, 8, 9, 10, 11, 12, or more than 12 populations of cells of increasing cell density from which fluorescence spectra are obtained, wherein the pluralities comprise populations of OD₆₀₀ of 5 and OD₆₀₀ of 25. In particular embodiments, the first and second pluralities comprise cell populations of OD₆₀₀ of 5, 10, 15, 20 and 25. In other embodiments, the first and second pluralities of cells comprise populations of OD₆₀₀ of 1 and 10, 1 and 15, 5 and 20, 10 and 20, or and 25. Preferably, cell density x and cell density 5x is within a dynamic range for spectrophotometric detection at 600 nm for a given cell type.

With regard to the water immiscible compound (WIC) for which selective spectral conditions are being sought, for purposes of determining the spectral conditions, the WIC may be added, for example, as a purified compound, to aqueous medium comprising cells of the first plurality. Alternatively, the cells of the first plurality may be recombinant cells modified to produce the WIC. In some embodiments utilizing recombinant cells producing WIC, the amount of WIC produced by the cell is previously established, for example, as a yield (grams of compound per gram of substrate, e.g., sucrose), a level of production (grams per liter) and/or a level of productivity (grams per liter per hour). In some embodiments where the first plurality comprises recombinant cells producing the WIC, the cells are cultured for a period of time sufficient for production of the WIC prior to determining spectral conditions specific for the WIC.

In some embodiments, each of the cell populations of the first plurality comprises the WIC in an equal amount. In other embodiments, the cell populations of the first plurality comprise WIC in differing amounts. Preferably, the amount of WIC is not in excess of the amount of fluorescent dye available to bind the WIC during said contacting. In some embodiments, each of the cell populations of the first plurality comprises WIC in an amount of at least 0.1 g/L. In other embodiments, each of the cell populations of the first plurality comprises WIC in an amount of 0.1 g/L to 10 g/l. In some embodiments, each of the populations of the first plurality comprise WIC in an amount of about 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0 or more than 15.0 g/L. In particular embodiments, the WIC is added to each of the populations of the first plurality as purified WIC, for example, in a solvent that does not contribute an appreciable amount of background fluorescence to the assay. In particular embodiments, WIC is exogenously added to each population of cells of the first plurality at a concentration of at least 2 g/L.

Preferably, the cells of the first and second pluralities are of the same cell type, so as to minimize any differences in the quantity or quality of endogenous cellular targets that may be bound by the fluorescent dye. Preferably, the cells of the second plurality do not comprise WIC, e.g., exogenously added or recombinantly produced WIC. However, where the WIC may be present in the cells of the second plurality as an endogenous molecule, the WIC will also be present in the cells of the first plurality as an endogenous molecule.

In some embodiments, at the excitation and/or emission wavelengths selected for the specific detection of WIC, the difference in fluorescence between a cell population from the first plurality (comprising WIC) and a cell population from the second plurality (not comprising WIC) having the same cell density is at least 80%. In some embodiments, the difference in fluorescence between these cell populations will be at least about 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500 or more than 500%.

In some embodiments, at the excitation and/or emission wavelengths selected for the specific detection of WIC, the difference in fluorescence between cell populations having cell density x and cell density 5x from the second plurality is no greater than 250%. In some embodiments, this difference is no greater than about 240, 230, 220, 210, 200, 190, 180, 170, 160, 150, 140, 130, 120, 110, 100, 90, 80, 70, 60, 50, 40, 20 or 10%.

The methods provided herein, useful for the determination of spectral conditions sufficient to selectively detect fluorescent dye bound to WIC produced from a plurality of cells, were applied towards the identification of spectral conditions suitable for the selective detection of Nile Red bound to farnesene in the presence of yeast cells. These results, provided below in Example 2, demonstrate that Nile Red bound to farnesene can be detected under spectral conditions where spillover of fluorescence from cell biomass is avoided. Accordingly, in another aspect, provided herein is a method of selectively detecting, in solution, farnesene produced from a cell, the method comprising: (a) contacting a solution with Nile Red, wherein the solution comprises a cell recombinantly producing farnesene; and (b) detecting Nile Red at an excitation wavelength of about 260 to 290 nm and an emission wavelength of about 530 to 570 nm.

Further provided herein are methods for determining spectral conditions that are selective for detecting autofluorescence from cells without influence from Nile-Red fluorescence, e.g. fluorescence from Nile Red bound to WIC. Autofluorescence can be used as a proxy for cell biomass, and thus, once spectral conditions that are selective for autofluorescence have been determined, WIC:cell biomass ratios for a given WIC-producing cell population can be obtained using two selective excitation/emission wavelength pairs.

In some embodiments, the method of determining spectral conditions selective for cell autofluorescence comprises:

(a) contacting the fluorescent dye with a first plurality of cell populations and a second plurality of cell populations, wherein cells of the first and second plurality are of the same cell type, wherein each plurality comprises a cell population having a cell density of x and a cell population having a cell density of 5x, wherein each of the cell populations of the first plurality comprise WIC, and the cell populations of the second plurality do not comprise WIC;

(b) determining an excitation spectrum for the first plurality and the second plurality, respectively; and

(c) selecting an excitation wavelength wherein:
(i) the difference in fluorescence between a cell population from the first plurality and a cell population from the second plurality having the same cell density is no greater than 80%; and
(ii) the difference in fluorescence between cell populations having cell density x and cell density 5× from the second plurality is at least 250%.

In some embodiments, the method of determining spectral conditions selective for cell autofluorescence comprises:

(a) contacting the fluorescent dye with a first plurality of cell populations and a second plurality of cell populations, wherein cells of the first and second plurality are of the same cell type, wherein each plurality comprises a cell population having a cell density of x and a cell population having a cell density of 5x, wherein each of the cell populations of the first plurality comprise WIC, and the cell populations of the second plurality do not comprise WIC;

(b) determining an emission spectrum for the first plurality and the second plurality, respectively; and

(c) selecting an emission wavelength wherein:
(i) the difference in fluorescence between a cell population from the first plurality and a cell population from the second plurality having the same cell density is no greater than 80%; and
(ii) the difference in fluorescence between cell populations having cell density x and cell density 5x from the second plurality is at least 250%.

In particular embodiments, the method of determining spectral conditions selective for cell autofluorescence comprises selecting both an excitation and emission wavelength, i.e., a compatible emission and excitation wavelength pairing, wherein (i) the difference in fluorescence between a cell population from the first plurality and a cell population from the second plurality having the same cell density is no greater than 80%; and (ii) the difference in fluorescence between cell populations having cell density x and cell density 5x from the second plurality is at least 250%.

In some embodiments, at the excitation and/or emission wavelengths selected for the specific detection of cell autofluorescence, the difference in fluorescence between a cell population from the first plurality (comprising WIC) and a cell population from the second plurality (not comprising WIC) having the same cell density is no greater than 80%. In some embodiments, the difference in fluorescence between these cell populations will be no greater than 75, 70, 65, 60, 55, 50, 45, 40, 35, 30, 25, 20, 15 or 10%.

In some embodiments, at the excitation and/or emission wavelengths selected for the specific detection of cell autofluorescence, the difference in fluorescence between cell populations having cell density x and cell density 5× from the second plurality is at least 250%. In some embodiments, this difference is at least 260, 270, 280, 290, 300, 310, 320, 330, 340, 350, 360, 370, 380, 390, 400, 410, 420, 430, 440, 450, 460, 470, 480, 490, 500 or more than 500%.

### 6.3 Methods of Screening

In another aspect, provided herein is a method of screening a library of cells for a cell or clonal population of cells recombinantly producing a water-immiscible compound, comprising: (a) contacting a solution with a fluorescent dye that directly binds the WIC, wherein the solution comprises a plurality of cells recombinantly producing the WIC; (b) detecting the fluorescent dye under spectral conditions suitable for the selective detection of the fluorescent dye bound to the recombinantly produced WIC; and (c) selecting a cell or clonal population of cells producing said recombinantly produced water-immiscible compound. In some embodiments, the method further comprises repeating said steps of detecting and selecting so that a water-immiscible compound producing cell or clonal population of cells is enriched over successive rounds of selection. In particular embodiments, the cell is a microbial cell genetically modified to produce one or more water-immiscible compounds at greater yield and/or with increased persistence compared to a parent microbial cell that is not genetically modified. In some embodiments, the methods of screening are sufficient to identify and select such a genetically modified microbial cell having increased water-immiscible compound production compared to a parent microbial cell that is not genetically modified.

In some embodiments, the method of screening is sufficient to identify a cell or clonal population of cells recombinantly producing one or more water-immiscible compounds expressed as a ratio of WIC to cell biomass. In such embodiments, the method of screening further comprises at step (b): determining a WIC:cell biomass ratio. In some embodiments, the cell biomass is determined by a method comprising detecting the autofluorescence of said plurality of cells under spectral conditions wherein fluorescence from the fluorescent dye bound to the WIC is not detected. The WIC:biomass ratio can be calculated based on the relative fluorescence units (RFU) of the separate yet specific measurements of WIC and biomass, respectively, utilizing select spectral conditions as described herein. In some embodiments, the method of screening is sufficient to identify a cell or clonal population of cells recombinantly producing one or more water-immiscible compounds in a WIC:biomass ratio of about 100:1, 95:1, 90:1, 85:1, 80:1, 75:1, 70:1, 65:1, 60:1, 55:1, 50:1, 45:1, 40:1, 35:1, 30:1, 25:1, 20:1, 15:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, 1:25, 1:30, 1:35, 1:40, 1:45, 1:50, 1:55, 1:60, 1:65, 1:70, 1:75, 1:80, 1:85, 1:90, 1:95 or 1:100. In some embodiments, the method of screening is sufficient to identify a cell or clonal population of cells recombinantly producing one or more water-immiscible compounds in a WIC:biomass ratio of greater than 100:1 or less than 1:100.

In some embodiments, the method of screening is sufficient to identify a cell or clonal population of cells recombinantly producing one or more water-immiscible compounds in an amount greater than about 10 grams per liter of fermentation medium. In some embodiments, the recombinantly produced water-immiscible compound is produced in an amount from about 10 to about 50 grams, more than about 15 grams, more than about 20 grams, more than about 25 grams, or more than about 30 grams per liter of cell culture.

In some embodiments, the method of screening is sufficient to identify a cell or clonal population of cells recombinantly producing one or more water-immiscible compounds in an amount greater than about 50 milligrams per gram of dry cell weight. In some embodiments, the recombinantly produced water-immiscible compound is produced in an amount from about 50 to about 1500 milligrams, more than about 100 milligrams, more than about 150 milligrams, more than about 200 milligrams, more than about 250 milligrams, more than about 500 milligrams, more than about 750 milligrams, or more than about 1000 milligrams per gram of dry cell weight.

In some embodiments, the method of screening is sufficient to identify a cell or clonal population of cells recombinantly producing one or more water-immiscible compounds in an amount that is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 2-fold, at least about 2.5-fold, at least about 5-fold, at least about 10-fold, at least about 20-fold, at least about 30-fold, at least about 40-fold, at least about 50-fold, at least about 75-fold, at least about 100-fold, at least about 200-fold, at least about 300-fold, at least about 400-fold, at least about 500-fold, or at least about 1,000-fold, or more, higher than the amount of the water-immiscible compound produced by a microbial cell that is not genetically modified as described herein, on a per unit volume of cell culture basis.

In some embodiments, the method of screening is sufficient to identify a cell or clonal population of cells recombinantly producing one or more water-immiscible compounds in an amount that is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 2-fold, at least about 2.5-fold, at least about 5-fold, at least about 10-fold, at least about 20-fold, at least about 30-fold, at least about 40-fold, at least about 50-fold, at least about 75-fold, at least about 100-fold, at least about 200-fold, at least about 300-fold, at least about 400-fold, at least about 500-fold, or at least about 1,000-fold, or more, higher than the amount of the water-immiscible compound produced by a microbial cell that is not genetically modified according to the methods provided herein, on a per unit dry cell weight basis.

In some embodiments, the method of screening is sufficient to identify a cell or clonal population of cells recombinantly producing one or more water-immiscible compounds in an amount that is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 2-fold, at least about 2.5-fold, at least about 5-fold, at least about 10-fold, at least about 20-fold, at least about 30-fold, at least about 40-fold, at least about 50-fold, at least about 75-fold, at least about 100-fold, at least about 200-fold, at least about 300-fold, at least about 400-fold, at least about 500-fold, or at least about 1,000-fold, or more, higher than the amount of the water-immiscible compound produced by a microbial cell that is not genetically modified according to the methods provided herein, on a per unit volume of cell culture per unit time basis.

In some embodiments, the method of screening is sufficient to identify a cell or clonal population of cells recombinantly producing one or more water-immiscible compounds in an amount that is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 2-fold, at least about 2.5-fold, at least about 5-fold, at least about 10-fold, at least about 20-fold, at least about 30-fold, at least about 40-fold, at least about 50-fold, at least about 75-fold, at least about 100-fold, at least about 200-fold, at least about 300-fold, at least about 400-fold, at least about 500-fold, or at least about 1,000-fold, or more, higher than the amount of the water-immiscible compound produced by a microbial cell that is not genetically modified according to the methods provided herein, on a per unit dry cell weight per unit time basis.

### 6.4 Host Cells and Recombinant Cells Producing WIC

In another aspect, provided herein is a cell or clonal cell population comprising one or more recombinantly produced water-immiscible compounds. Cells useful in the methods and compositions provided herein include any cell capable of naturally or recombinantly producing a water-immiscible compound, e.g., an isoprenoid, a polyketide, a fatty acid, and the like. In some embodiments, the cell is a prokaryotic cell. In some embodiments, the cell is a bacterial cell. In some embodiments, the cell is an Escherichia coli cell. In some embodiments, the cell is a eukaryotic cell. In some embodiments, the cell is a mammalian cell. In some embodiments, the cell is a Chinese hamster ovary (CHO) cell, a COS-7 cell, a mouse fibroblast cell, a mouse embryonal carcinoma cell, or a mouse embryonic stem cell. In some embodiments, the cell is an insect cell. In some embodiments, the cell is a S2 cell, a Schneider cell, a S12 cell, a 5B1-4 cell, a Tn5 cell, or a Sf9 cell. In some embodiments, the cell is a unicellular eukaryotic organism cell.

In some embodiments, the cell is a mycelial bacterial cell. In some embodiments, the mycelial bacterial cell is of the class actinomycetes. In particular embodiments, the mycelial bacterial cell is of the genera Streptomyces, for example, Streptomyces ambofaciens, Streptomyces avermitilis, Streptomyces azureus, Streptomyces cinnamonensis, Streptomyces coelicolor, Streptomyces curacoi, Streptomyces erythraeus, Streptomyces fradiae, Streptomyces galilaeus, Streptomyces glaucescens, Streptomyces hygroscopicus, Streptomyces lividans, Streptomyces parvulus, Streptomyces peucetius, Streptomyces rimosus, Streptomyces roseofulvus, Streptomyces thermotolerans, Streptomyces violaceoruber.

In another embodiment, the cell is a fungal cell. In a more particular embodiment, the cell is a yeast cell. Yeasts useful in the methods and compositions provided herein include yeasts that have been deposited with microorganism depositories (e.g. IFO, ATCC, etc.) and belong to the genera Aciculoconidium, Ambrosiozyma, Arthroascus, Arxiozyma, Ashbya, Babjevia, Bensingtonia, Botryoascus, Botryozyma, Brettanomyces, Bullera, Bulleromyces, Candida, Citeromyces, Clavispora, Cryptococcus, Cystofilobasidium, Debaryomyces, Dekkara, Dipodascopsis, Dipodascus, Eeniella, Endomycopsella, Eremascus, Eremothecium, Erythrobasidium, Fellomyces, Filobasidium, Galactomyces, Geotrichum, Guilliermondella, Hanseniaspora, Hansenula, Hasegawaea, Holtermannia, Hormoascus, Hyphopichia, Issatchenkia, Kloeckera, Kloeckeraspora, Kluyveromyces, Kondoa, Kuraishia, Kurtzmanomyces, Leucosporidium, Lipomyces, Lodderomyces, Malassezia, Metschnikowia, Mrakia, Myxozyma, Nadsonia, Nakazawaea, Nematospora, Ogataea, Oosporidium, Pachysolen, Phachytichospora, Phaffia, Pichia, Rhodosporidium, Rhodotorula, Saccharomyces, Saccharomycodes, Saccharomycopsis, Saitoella, Sakaguchia, Saturnospora, Schizoblastosporion, Schizosaccharomyces, Schwanniomyces, Sporidiobolus, Sporobolomyces, Sporopachydermia, Stephanoascus, Sterigmatomyces, Sterigmatosporidium, Symbiotaphrina, Sympodiomyces, Sympodiomycopsis, Torulaspora, Trichosporiella, Trichosporon, Trigonopsis, Tsuchiyaea, Udeniomyces, Waltomyces, Wickerhamia, Wickerhamiella, Williopsis, Yamadazyma, Yarrowia, Zygoascus, Zygosaccharomyces, Zygowilliopsis, and Zygozyma, among others.

In particular embodiments, useful yeasts in the methods and compositions provided herein include Saccharomyces cerevisiae, Pichia pastoris, Schizosaccharomyces pombe, Dekkera bruxellensis, Kluyveromyces lactis (previously called Saccharomyces lactis), Kluveromyces marxianus, Arxula adeninivorans, or Hansenula polymorpha (now known as Pichia angusta). In some embodiments, the microbe is a strain of the genus Candida, such as Candida lipolytica, Candida guilliermondii, Candida krusei, Candida pseudotropicalis, or Candida utilis.

In a particular embodiment, the cell is a Saccharomyces cerevisiae cell. In some embodiments, the strain of the Saccharomyces cerevisiae cell is selected from the group consisting of Baker's yeast, CBS 7959, CBS 7960, CBS 7961, CBS 7962, CBS 7963, CBS 7964, IZ-1904, TA, BG-1, CR-1, SA-1, M-26, Y-904, PE-2, PE-5, VR-1, BR-1, BR-2, ME-2, VR-2, MA-3, MA-4, CAT-1, CB-1, NR-1, BT-1, and AL-1. In some embodiments, the strain of Saccharomyces cerevisiae is selected from the group consisting of PE-2, CAT-1, VR-1, BG-1, CR-1, and SA-1. In a particular embodiment, the strain of Saccharomyces cerevisiae is PE-2. In another particular embodiment, the strain of Saccharomyces cerevisiae is CAT-1. In another particular embodiment, the strain of Saccharomyces cerevisiae is BG-1.

In some embodiments, the cell is a haploid microbial cell. In other embodiments, the cell is a diploid microbial cell. In some embodiments, the cell is heterozygous. In other embodiments, the cell is homozygous other than for its mating type allele (i.e., if the cell should sporulate, the resulting four haploid microbial cells would be genetically identical except for their mating type allele, which in two of the haploid cells would be mating type a and in the other two haploid cells would be mating type alpha).

In some embodiments, the cell is a cell that is suitable for industrial fermentation, e.g., bioethanol fermentation. In particular embodiments, the cell is conditioned to subsist under high solvent concentration, high temperature, expanded substrate utilization, nutrient limitation, osmotic stress due, acidity, sulfite and bacterial contamination, or combinations thereof, which are recognized stress conditions of the industrial fermentation environment.

Exemplary water-immiscible compound producing cells, e.g., cells recombinantly producing isoprenoids, polyketides, and fatty acids, and methods for generating such cells, are provided below.

6.4.1 Recombinant Cells Producing Isoprenoids

In one aspect, provided herein are methods of detecting isoprenoid production in a cell or a clonal population of cells, e.g., genetically modified to recombinantly produce one or more isoprenoid compounds. Isoprenoids are derived from isopentenyl pyrophosphate (IPP), which can be biosynthesized by enzymes of the mevalonate-dependent (“MEV”) pathway or the 1-deoxy-D-xylulose 5-diphosphate (“DXP”) pathway. A schematic representation of the MEV pathway is described in FIG. 1A, and a schematic representation of the DXP pathway is described in FIG. 1B.

6.4.1.1 MEV Pathway

In some embodiments of the methods of detecting an isoprenoid producing cell provided herein, the isoprenoid producing cell comprises one or more heterologous nucleotide sequences encoding one or more enzymes of the MEV pathway, which effects increased production of one or more isoprenoid compounds as compared to a genetically unmodified parent cell.

In some embodiments, the isoprenoid producing cell comprises a heterologous nucleotide sequence encoding an enzyme that can condense two molecules of acetyl-coenzyme A to form acetoacetyl-CoA, e.g., an acetyl-CoA thiolase. Illustrative examples of nucleotide sequences encoding such an enzyme include, but are not limited to: (NC_{—}000913 REGION: 2324131.2325315; Escherichia coli), (D49362; Paracoccus denitrificans), and (L20428; Saccharomyces cerevisiae).

In some embodiments, the isoprenoid producing cell comprises a heterologous nucleotide sequence encoding an enzyme that can condense acetoacetyl-CoA with another molecule of acetyl-CoA to form 3-hydroxy-3-methylglutaryl-CoA (HMG-CoA), e.g., a HMG-CoA synthase. Illustrative examples of nucleotide sequences encoding such an enzyme include, but are not limited to: (NC_{—}001145. complement 19061.20536; Saccharomyces cerevisiae), (X96617; Saccharomyces cerevisiae), (X83882; Arabidopsis thaliana), (AB037907; Kitasatospora griseola), (BT007302; Homo sapiens), and (NC_{—}002758, Locus tag SAV2546, GeneID 1122571; Staphylococcus aureus).

In some embodiments, the isoprenoid producing cell comprises a heterologous nucleotide sequence encoding an enzyme that can convert HMG-CoA into mevalonate, e.g., a HMG-CoA reductase. Illustrative examples of nucleotide sequences encoding such an enzyme include, but are not limited to: (NM_{—}206548; Drosophila melanogaster), (NC_{—}002758, Locus tag SAV2545, GeneID 1122570; Staphylococcus aureus), (NM_{—}204485; Gallus gallus), (AB015627; Streptomyces sp. KO 3988), (AF542543; Nicotiana attenuata), (AB037907; Kitasatospora griseola), (AX128213, providing the sequence encoding a truncated HMGR; Saccharomyces cerevisiae), and (NC_{—}001145: complement (115734.118898; Saccharomyces cerevisiae).

In some embodiments, the isoprenoid producing cell comprises a heterologous nucleotide sequence encoding an enzyme that can convert mevalonate into mevalonate 5-phosphate, e.g., a mevalonate kinase. Illustrative examples of nucleotide sequences encoding such an enzyme include, but are not limited to: (L77688; Arabidopsis thaliana), and (X55875; Saccharomyces cerevisiae).

In some embodiments, the isoprenoid producing cell comprises a heterologous nucleotide sequence encoding an enzyme that can convert mevalonate 5-phosphate into mevalonate 5-pyrophosphate, e.g., a phosphomevalonate kinase. Illustrative examples of nucleotide sequences encoding such an enzyme include, but are not limited to: (AF429385; Hevea brasiliensis), (NM_{—}006556; Homo sapiens), and (NC_{—}001145. complement 712315.713670; Saccharomyces cerevisiae).

In some embodiments, the isoprenoid producing cell comprises a heterologous nucleotide sequence encoding an enzyme that can convert mevalonate 5-pyrophosphate into IPP, e.g., a mevalonate pyrophosphate decarboxylase. Illustrative examples of nucleotide sequences encoding such an enzyme include, but are not limited to: (X97557; Saccharomyces cerevisiae), (AF290095; Enterococcus faecium), and (U49260; Homo sapiens).

In some embodiments, the isoprenoid producing cell comprises one or more heterologous nucleotide sequences encoding more than one enzyme of the MEV pathway. In some embodiments, the isoprenoid producing cell comprises one or more heterologous nucleotide sequences encoding two enzymes of the MEV pathway. In some embodiments, the isoprenoid producing cell comprises one or more heterologous nucleotide sequences encoding an enzyme that can convert HMG-CoA into mevalonate and an enzyme that can convert mevalonate into mevalonate 5-phosphate. In some embodiments, the isoprenoid producing cell comprises one or more heterologous nucleotide sequences encoding three enzymes of the MEV pathway. In some embodiments, the isoprenoid producing cell comprises one or more heterologous nucleotide sequences encoding four enzymes of the MEV pathway. In some embodiments, the isoprenoid producing cell comprises one or more heterologous nucleotide sequences encoding five enzymes of the MEV pathway. In some embodiments, the isoprenoid producing cell comprises one or more heterologous nucleotide sequences encoding six enzymes of the MEV pathway.

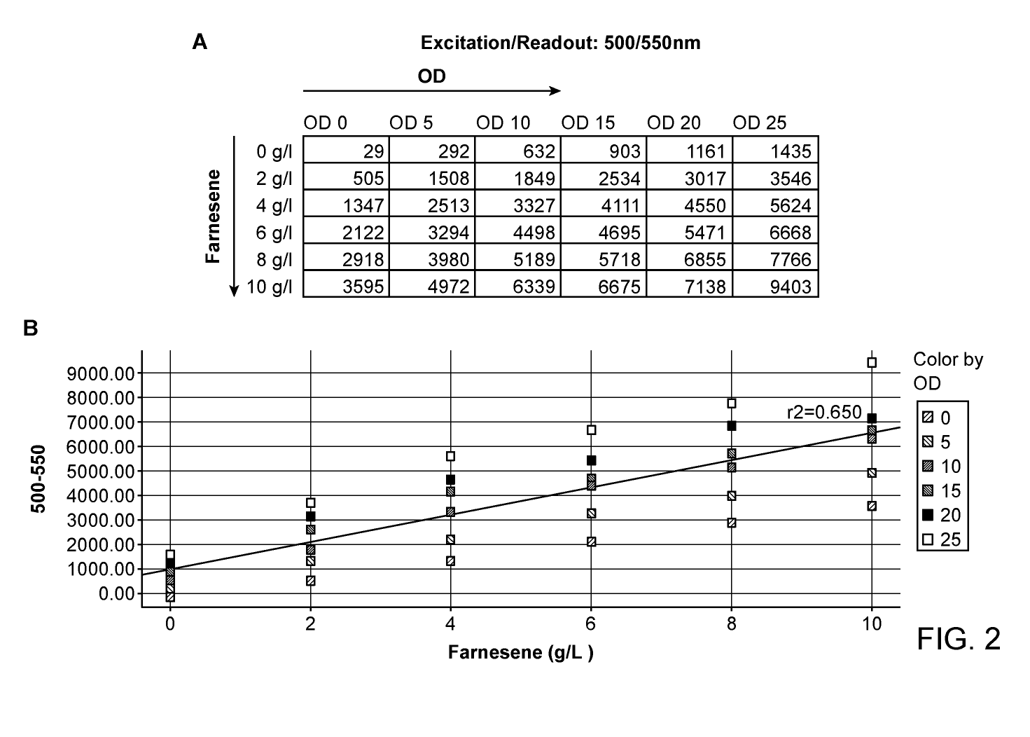

In some embodiments, the isoprenoid producing cell further comprises a heterologous nucleotide sequence encoding an enzyme that can convert IPP generated via the MEV pathway into its isomer, dimethylallyl pyrophosphate (“DMAPP”). DMAPP can be condensed and modified through the action of various additional enzymes to form simple and more complex isoprenoids (FIG. 2).

6.4.1.2 DXP Pathway

In some embodiments of the methods of detecting an isoprenoid producing cell provided herein, the isoprenoid producing cell comprises one or more heterologous nucleotide sequences encoding one or more enzymes of the DXP pathway, which effects increased production of one or more isoprenoid compounds as compared to a genetically unmodified parent cell.

In some embodiments, the isoprenoid producing cell comprises a heterologous nucleotide sequence encoding an enzyme that can condense two molecules of acetyl-coenzyme A to form acetoacetyl-CoA, e.g., an acetyl-CoA thiolase. Illustrative examples of nucleotide sequences encoding such an enzyme include, but are not limited to: (NC_{—}000913 REGION: 2324131.2325315; Escherichia coli), (D49362; Paracoccus denitrificans), and (L20428; Saccharomyces cerevisiae).

In some embodiments, the isoprenoid producing cell comprises a heterologous nucleotide sequence encoding an enzyme, e.g., 1-deoxy-D-xylulose-5-phosphate synthase, that can condense pyruvate with D-glyceraldehyde 3-phosphate to make 1-deoxy-D-xylulose-5-phosphate. Illustrative examples of nucleotide sequences encoding such an enzyme include but are not limited to: (AF035440; Escherichia coli), (NC_{—}002947, locus tag PP0527; Pseudomonas putida KT2440), (CP000026, locus tag SPA2301; Salmonella enterica Paratyphi, see ATCC 9150), (NC_{—}007493, locus tag RSP_{—}0254; Rhodobacter sphaeroides 2.4.1), (NC_{—}005296, locus tag RPA0952; Rhodopseudomonas palustris CGA009), (NC_{—}004556, locus tag PD1293; Xylella fastidiosa Temeculal), and (NC_{—}003076, locus tag AT5G11380; Arabidopsis thaliana).

In some embodiments, the isoprenoid producing cell comprises a heterologous nucleotide sequence encoding an enzyme, e.g., 1-deoxy-D-xylulose-5-phosphate reductoisomerase, that can convert 1-deoxy-D-xylulose-5-phosphate to 2C-methyl-D-erythritol-4-phosphate. Illustrative examples of nucleotide sequences include but are not limited to: (AB013300; Escherichia coli), (AF148852; Arabidopsis thaliana), (NC_{—}002947, locus tag PP1597; Pseudomonas putida KT2440), (AL939124, locus tag SCO5694; Streptomyces coelicolor A3(2)), (NC_{—}007493, locus tag RSP_{—}2709; Rhodobacter sphaeroides 2.4.1), and (NC_{—}007492, locus tag Pfl_{—}1107; Pseudomonas fluorescens PfO-1).

In some embodiments, the isoprenoid producing cell comprises a heterologous nucleotide sequence encoding an enzyme, e.g., 4-diphosphocytidyl-2C-methyl-D-erythritol synthase, that can convert 2C-methyl-D-erythritol-4-phosphate to 4-diphosphocytidyl-2C-methyl-D-erythritol. Illustrative examples of nucleotide sequences include but are not limited to: (AF230736; Escherichia coli), (NC_{—}007493, locus tag RSP_{—}2835; Rhodobacter sphaeroides 2.4.1), (NC_{—}003071, locus tag AT2G02500; Arabidopsis thaliana), and (NC_{—}002947, locus tag PP1614; Pseudomonas putida KT2440).

In some embodiments, the isoprenoid producing cell comprises a heterologous nucleotide sequence encoding an enzyme, e.g., 4-diphosphocytidyl-2C-methyl-D-erythritol kinase, that can convert 4-diphosphocytidyl-2C-methyl-D-erythritol to 4-diphosphocytidyl-2C-methyl-D-erythritol-2-phosphate. Illustrative examples of nucleotide sequences include but are not limited to: (AF216300; Escherichia coli) and (NC_{—}007493, locus tag RSP_{—}1779; Rhodobacter sphaeroides 2.4.1).

In some embodiments, the isoprenoid producing cell comprises a heterologous nucleotide sequence encoding an enzyme, 2C-methyl-D-erythritol 2,4-cyclodiphosphate synthase, that can convert 4-diphosphocytidyl-2C-methyl-D-erythritol-2-phosphate to 2C-methyl-D-erythritol 2,4-cyclodiphosphate. Illustrative examples of nucleotide sequences include but are not limited to: (AF230738; Escherichia coli), (NC_{—}007493, locus tag RSP_{—}6071; Rhodobacter sphaeroides 2.4.1), and (NC_{—}002947, locus tag PP1618; Pseudomonas putida KT2440).

In some embodiments, the isoprenoid producing cell comprises a heterologous nucleotide sequence encoding an enzyme, e.g., 1-hydroxy-2-methyl-2-(E)-butenyl-4-diphosphate synthase, that can convert 2C-methyl-D-erythritol 2,4-cyclodiphosphate to 1-hydroxy-2-methyl-2-(E)-butenyl-4-diphosphate. Illustrative examples of nucleotide sequences include but are not limited to: (AY033515; Escherichia coli), (NC_{—}002947, locus tag PP0853; Pseudomonas putida KT2440), and (NC_{—}007493, locus tag RSP_{—}2982; Rhodobacter sphaeroides 2.4.1).

In some embodiments, the isoprenoid producing cell comprises a heterologous nucleotide sequence encoding an enzyme, e.g., isopentyl/dimethylallyl diphosphate synthase, that can convert 1-hydroxy-2-methyl-2-(E)-butenyl-4-diphosphate into either IPP or its isomer, DMAPP. Illustrative examples of nucleotide sequences include but are not limited to: (AY062212; Escherichia coli) and (NC_{—}002947, locus tag PP0606; Pseudomonas putida KT2440).

In some embodiments, the isoprenoid producing cell comprises one or more heterologous nucleotide sequences encoding more than one enzyme of the DXP pathway. In some embodiments, the isoprenoid producing cell comprises one or more heterologous nucleotide sequences encoding two enzymes of the DXP pathway. In some embodiments, the isoprenoid producing cell comprises one or more heterologous nucleotide sequences encoding three enzymes of the DXP pathway. In some embodiments, the isoprenoid producing cell comprises one or more heterologous nucleotide sequences encoding four enzymes of the DXP pathway. In some embodiments, the isoprenoid producing cell comprises one or more heterologous nucleotide sequences encoding five enzymes of the DXP pathway. In some embodiments, the isoprenoid producing cell comprises one or more heterologous nucleotide sequences encoding six enzymes of the DXP pathway. In some embodiments, the isoprenoid producing cell comprises one or more heterologous nucleotide sequences encoding five enzymes of the DXP pathway. In some embodiments, the isoprenoid producing cell comprises one or more heterologous nucleotide sequences encoding seven enzymes of the DXP pathway.

In some embodiments, “cross talk” (or interference) between the host cell's own metabolic processes and those processes involved with the production of IPP are minimized or eliminated entirely. For example, cross talk is minimized or eliminated entirely when the host microorganism relies exclusively on the DXP pathway for synthesizing IPP, and a MEV pathway is introduced to provide additional IPP. Such a host organism would not be equipped to alter the expression of the MEV pathway enzymes or process the intermediates associated with the MEV pathway. Organisms that rely exclusively or predominately on the DXP pathway include, for example, Escherichia coli.

In some embodiments, the host cell produces IPP via the MEV pathway, either exclusively or in combination with the DXP pathway. In other embodiments, a host's DXP pathway is functionally disabled so that the host cell produces IPP exclusively through a heterologously introduced MEV pathway. The DXP pathway can be functionally disabled by disabling gene expression or inactivating the function of one or more of the DXP pathway enzymes.

In some embodiments, the isoprenoid produced by the cell is a C₅ isoprenoid. These compounds are derived from one isoprene unit and are also called hemiterpenes. An illustrative example of a hemiterpene is isoprene. In other embodiments, the isoprenoid is a C₁₀ isoprenoid. These compounds are derived from two isoprene units and are also called monoterpenes. Illustrative examples of monoterpenes are limonene, citranellol, geraniol, menthol, perillyl alcohol, linalool, thujone, and myrcene. In other embodiments, the isoprenoid is a C₁₅ isoprenoid. These compounds are derived from three isoprene units and are also called sesquiterpenes. Illustrative examples of sesquiterpenes are periplanone B, gingkolide B, amorphadiene, artemisinin, artemisinic acid, valencene, nootkatone, epi-cedrol, epi-aristolochene, farnesol, gossypol, sanonin, periplanone, forskolin, and patchoulol (which is also known as patchouli alcohol). In other embodiments, the isoprenoid is a C₂₀ isoprenoid. These compounds are derived from four isoprene units and also called diterpenes. Illustrative examples of diterpenes are casbene, eleutherobin, paclitaxel, prostratin, pseudopterosin, and taxadiene. In yet other examples, the isoprenoid is a C₂₀₊ isoprenoid. These compounds are derived from more than four isoprene units and include: triterpenes (C₃₀ isoprenoid compounds derived from 6 isoprene units) such as arbrusideE, bruceantin, testosterone, progesterone, cortisone, digitoxin, and squalene; tetraterpenes (C₄₀ isoprenoid compounds derived from 8 isoprenoids) such as β-carotene; and polyterpenes (C₄₀₊ isoprenoid compounds derived from more than 8 isoprene units) such as polyisoprene. In some embodiments, the isoprenoid is selected from the group consisting of abietadiene, amorphadiene, carene, α-farnesene, β-farnesene, farnesol, geraniol, geranylgeraniol, isoprene, linalool, limonene, myrcene, nerolidol, ocimene, patchoulol, β-pinene, sabinene, γ-terpinene, terpinolene and valencene. Isoprenoid compounds also include, but are not limited to, carotenoids (such as lycopene, α- and β-carotene, α- and β-cryptoxanthin, bixin, zeaxanthin, astaxanthin, and lutein), steroid compounds, and compounds that are composed of isoprenoids modified by other chemical groups, such as mixed terpene-alkaloids, and coenzyme Q-10.

In some embodiments, the isoprenoid producing cell further comprises a heterologous nucleotide sequence encoding an enzyme that can convert IPP generated via the MEV pathway into DMAPP, e.g., an IPP isomerase. Illustrative examples of nucleotide sequences encoding such an enzyme include, but are not limited to: (NC_{—}000913, 3031087.3031635; Escherichia coli), and (AF082326; Haematococcus pluvialis).

In some embodiments, the isoprenoid producing cell further comprises a heterologous nucleotide sequence encoding a polyprenyl synthase that can condense IPP and/or DMAPP molecules to form polyprenyl compounds containing more than five carbons.

In some embodiments, the isoprenoid producing cell comprises a heterologous nucleotide sequence encoding an enzyme that can condense one molecule of IPP with one molecule of DMAPP to form one molecule of geranyl pyrophosphate (“GPP”), e.g., a GPP synthase. Illustrative examples of nucleotide sequences encoding such an enzyme include, but are not limited to: (AF513111; Abies grandis), (AF513112; Abies grandis), (AF513113; Abies grandis), (AY534686; Antirrhinum majus), (AY534687; Antirrhinum majus), (Y17376; Arabidopsis thaliana), (AE016877, Locus AP 11092; Bacillus cereus; ATCC 14579), (AJ243739; Citrus sinensis), (AY534745; Clarkia breweri), (AY953508; Ips pini), (DQ286930; Lycopersicon esculentum), (AF182828; Mentha×piperita), (AF182827; Mentha×piperita), (MPI249453; Mentha×piperita), (PZE431697, Locus CAD24425; Paracoccus zeaxanthinifaciens), (AY866498; Picrorhiza kurrooa), (AY351862; Vitis vinifera), and (AF203881, Locus AAF12843; Zymomonas mobilis).

In some embodiments, the isoprenoid producing cell comprises a heterologous nucleotide sequence encoding an enzyme that can condense two molecules of IPP with one molecule of DMAPP, or add a molecule of IPP to a molecule of GPP, to form a molecule of farnesyl pyrophosphate (“FPP”), e.g., a FPP synthase. Illustrative examples of nucleotide sequences that encode such an enzyme include, but are not limited to: (ATU80605; Arabidopsis thaliana), (ATHFPS2R; Arabidopsis thaliana), (AAU36376; Artemisia annua), (AF461050; Bos taurus), (D00694; Escherichia coli K-12), (AE009951, Locus AAL95523; Fusobacterium nucleatum subsp. nucleatum ATCC 25586), (GFFPPSGEN; Gibberella fujikuroi), (CP000009, Locus AAW60034; Gluconobacter oxydans 621H), (AF019892; Helianthus annuus), (HUMFAPS; Homo sapiens), (KLPFPSQCR; Kluyveromyces lactic), (LAU15777; Lupinus albus), (LAU20771; Lupinus albus), (AF309508; Mus musculus), (NCFPPSGEN; Neurospora crassa), (PAFPS1; Parthenium argentatum), (PAFPS2; Parthenium argentatum), (RATFAPS; Rattus norvegicus), (YSCFPP; Saccharomyces cerevisiae), (D89104; Schizosaccharomyces pombe), (CP000003, Locus AAT87386; Streptococcus pyogenes), (CP000017, Locus AAZ51849; Streptococcus pyogenes), (NC_{—}008022, Locus YP_{—}598856; Streptococcus pyogenes MGAS 10270), (NC_{—}008023, Locus YP_{—}600845; Streptococcus pyogenes MGAS2096), (NC_{—}008024, Locus YP_{—}602832; Streptococcus pyogenes MGAS 10750), (MZEFPS; Zea mays), (AE000657, Locus AAC06913; Aquifex aeolicus VF5), (NM_{—}202836; Arabidopsis thaliana), (D84432, Locus BAA12575; Bacillus subtilis), (U12678, Locus AAC28894; Bradyrhizobium japonicum USDA 110), (BACFDPS; Geobacillus stearothermophilus), (NC_{—}002940, Locus NP_{—}873754; Haemophilus ducreyi 35000HP), (L42023, Locus AAC23087; Haemophilus influenzae Rd KW20), (J05262; Homo sapiens), (YP_{—}395294; Lactobacillus sakei subsp. sakei 23K), (NC_{—}005823, Locus YP_{—}000273; Leptospira interrogans serovar Copenhageni str. Fiocruz L1-130), (AB003187; Micrococcus luteus), (NC_{—}002946, Locus YP_{—}208768; Neisseria gonorrhoeae FA 1090), (U00090, Locus AAB91752; Rhizobium sp. NGR234), (J05091; Saccharomyces cerevisae), (CP000031, Locus AAV93568; Silicibacter pomeroyi DSS-3), (AE008481, Locus AAK99890; Streptococcus pneumoniae R6), and (NC_{—}004556, Locus NP 779706; Xylella fastidiosa Temeculal).

In some embodiments, the isoprenoid producing cell further comprises a heterologous nucleotide sequence encoding an enzyme that can combine IPP and DMAPP or IPP and FPP to form geranylgeranyl pyrophosphate (“GGPP”). Illustrative examples of nucleotide sequences that encode such an enzyme include, but are not limited to: (ATHGERPYRS; Arabidopsis thaliana), (BT005328; Arabidopsis thaliana), (NM_{—}119845; Arabidopsis thaliana), (NZ_AAJM01000380, Locus ZP_{—}00743052; Bacillus thuringiensis serovar israelensis, ATCC 35646 sq1563), (CRGGPPS; Catharanthus roseus), (NZ_AABF02000074, Locus ZP_{—}00144509; Fusobacterium nucleatum subsp. vincentii, ATCC 49256), (GFGGPPSGN; Gibberella fujikuroi), (AY371321; Ginkgo biloba), (AB055496; Hevea brasiliensis), (AB017971; Homo sapiens), (MCI276129; Mucor circinelloides f. lusitanicus), (AB016044; Mus musculus), (AABX01000298, Locus NCU01427; Neurospora crassa), (NCU20940; Neurospora crassa), (NZ_AAKL01000008, Locus ZP_{—}00943566; Ralstonia solanacearum UW551), (AB118238; Rattus norvegicus), (SCU31632; Saccharomyces cerevisiae), (AB016095; Synechococcus elongates), (SAGGPS; Sinapis alba), (SSOGDS; Sulfolobus acidocaldarius), (NC_{—}007759, Locus YP_{—}461832; Syntrophus aciditrophicus SB), (NC_{—}006840, Locus YP_{—}204095; Vibrio fischeri ES 114), (NM_{—}112315; Arabidopsis thaliana), (ERWCRTE; Pantoea agglomerans), (D90087, Locus BAA14124; Pantoea ananatis), (X52291, Locus CAA36538; Rhodobacter capsulatus), (AF195122, Locus AAF24294; Rhodobacter sphaeroides), and (NC_{—}004350, Locus NP_{—}721015; Streptococcus mutans UA159).

In some embodiments, the isoprenoid producing cell further comprises a heterologous nucleotide sequence encoding an enzyme that can modify a polyprenyl to form a hemiterpene, a monoterpene, a sesquiterpene, a diterpene, a triterpene, a tetraterpene, a polyterpene, a steroid compound, a carotenoid, or a modified isoprenoid compound.

In some embodiments, the heterologous nucleotide encodes a carene synthase. Illustrative examples of suitable nucleotide sequences include, but are not limited to: (AF461460, REGION 43.1926; Picea abies) and (AF527416, REGION: 78.1871; Salvia stenophylla).

In some embodiments, the heterologous nucleotide encodes a geraniol synthase. Illustrative examples of suitable nucleotide sequences include, but are not limited to: (AJ457070; Cinnamomum tenuipilum), (AY362553; Ocimum basilicum), (DQ234300; Perilla frutescens strain 1864), (DQ234299; Perilla citriodora strain 1861), (DQ234298; Perilla citriodora strain 4935), and (DQ088667; Perilla citriodora).

In some embodiments, the heterologous nucleotide encodes a linalool synthase. Illustrative examples of a suitable nucleotide sequence include, but are not limited to: (AF497485; Arabidopsis thaliana), (AC002294, Locus AAB71482; Arabidopsis thaliana), (AY059757; Arabidopsis thaliana), (NM_{—}104793; Arabidopsis thaliana), (AF154124; Artemisia annua), (AF067603; Clarkia breweri), (AF067602; Clarkia concinna), (AF067601; Clarkia breweri), (U58314; Clarkia breweri), (AY840091; Lycopersicon esculentum), (DQ263741; Lavandula angustifolia), (AY083653; Mentha citrate), (AY693647; Ocimum basilicum), (XM_{—}463918; Oryza sativa), (AP004078, Locus BAD07605; Oryza sativa), (XM_{—}463918, Locus XP_{—}463918; Oryza sativa), (AY917193; Perilla citriodora), (AF271259; Perilla frutescens), (AY473623; Picea abies), (DQ195274; Picea sitchensis), and (AF444798; Perilla frutescens var. crispa cultivar No. 79).

In some embodiments, the heterologous nucleotide encodes a limonene synthase. Illustrative examples of suitable nucleotide sequences include, but are not limited to: (+)-limonene synthases (AF514287, REGION: 47.1867; Citrus limon) and (AY055214, REGION: 48.1889; Agastache rugosa) and (−)-limonene synthases (DQ195275, REGION: 1.1905; Picea sitchensis), (AF006193, REGION: 73.1986; Abies grandis), and (MHC4SLSP, REGION: 29.1828; Mentha spicata).

In some embodiments, the heterologous nucleotide encodes a myrcene synthase. Illustrative examples of suitable nucleotide sequences include, but are not limited to: (U87908; Abies grandis), (AY195609; Antirrhinum majus), (AY195608; Antirrhinum majus), (NM_{—}127982; Arabidopsis thaliana TPS 10), (NM_{—}113485; Arabidopsis thaliana ATTPS-CIN), (NM_{—}113483; Arabidopsis thaliana ATTPS-CIN), (AF271259; Perilla frutescens), (AY473626; Picea abies), (AF369919; Picea abies), and (AJ304839; Quercus ilex).

In some embodiments, the heterologous nucleotide encodes a ocimene synthase. Illustrative examples of suitable nucleotide sequences include, but are not limited to: (AY195607; Antirrhinum majus), (AY195609; Antirrhinum majus), (AY195608; Antirrhinum majus), (AK221024; Arabidopsis thaliana), (NM_{—}113485; Arabidopsis thaliana ATTPS-CIN), (NM_{—}113483; Arabidopsis thaliana ATTPS-CIN), (NM_{—}117775; Arabidopsis thaliana ATTPS03), (NM_{—}001036574; Arabidopsis thaliana ATTPS03), (NM_{—}127982; Arabidopsis thaliana TPS 10), (AB 110642; Citrus unshiu CitMTSL4), and (AY575970; Lotus corniculatus var. japonicus).

In some embodiments, the heterologous nucleotide encodes an α-pinene synthase. Illustrative examples of suitable nucleotide sequences include, but are not limited to: (+) α-pinene synthase (AF543530, REGION: 1.1887; Pinus taeda), (−)α-pinene synthase (AF543527, REGION: 32.1921; Pinus taeda), and (+)/(−)α-pinene synthase (AGU87909, REGION: 6111892; Abies grandis).

In some embodiments, the heterologous nucleotide encodes a β-pinene synthase. Illustrative examples of suitable nucleotide sequences include, but are not limited to: (−)β-pinene synthases (AF276072, REGION: 1.1749; Artemisia annua) and (AF514288, REGION: 26.1834; Citrus limon).

In some embodiments, the heterologous nucleotide encodes a sabinene synthase. An illustrative example of a suitable nucleotide sequence includes but is not limited to AF051901, REGION: 26.1798 from Salvia officinalis.

In some embodiments, the heterologous nucleotide encodes a γ-terpinene synthase. Illustrative examples of suitable nucleotide sequences include: (AF514286, REGION: 30.1832 from Citrus limon) and (AB 110640, REGION 1.1803 from Citrus unshiu).

In some embodiments, the heterologous nucleotide encodes a terpinolene synthase. Illustrative examples of a suitable nucleotide sequence include but are not limited to: (AY693650 from Oscimum basilicum) and (AY906866, REGION: 10.1887 from Pseudotsuga menziesii).

In some embodiments, the heterologous nucleotide encodes an amorphadiene synthase. An illustrative example of a suitable nucleotide sequence is SEQ ID NO. 37 of U.S. Patent Publication No. 2004/0005678.

In some embodiments, the heterologous nucleotide encodes a α-farnesene synthase. Illustrative examples of suitable nucleotide sequences include, but are not limited to DQ309034 from Pyrus communis cultivar d'Anjou (pear; gene name AFS 1) and AY182241 from Malus domestica (apple; gene AFS1). Pechouus et al., Planta 219(1):84-94 (2004).

In some embodiments, the heterologous nucleotide encodes a β-farnesene synthase. Illustrative examples of suitable nucleotide sequences include but are not limited to GenBank accession number AF024615 from Mentha×piperita (peppermint; gene Tspa11), and AY835398 from Artemisia annua. Picaud et al., Phytochemistry 66(9): 961-967 (2005).

In some embodiments, the heterologous nucleotide encodes a farnesol synthase. Illustrative examples of suitable nucleotide sequences include, but are not limited to GenBank accession number AF529266 from Zea mays and YDR481C from Saccharomyces cerevisiae (gene Pho8). Song, L., Applied Biochemistry and Biotechnology 128:149-158 (2006).

In some embodiments, the heterologous nucleotide encodes a nerolidol synthase. An illustrative example of a suitable nucleotide sequence includes, but is not limited to AF529266 from Zea mays (maize; gene tps1).

In some embodiments, the heterologous nucleotide encodes a patchouliol synthase. Illustrative examples of suitable nucleotide sequences include, but are not limited to AY508730 REGION: 1.1659 from Pogostemon cablin.

In some embodiments, the heterologous nucleotide encodes a nootkatone synthase. Illustrative examples of a suitable nucleotide sequence include, but are not limited to AF441124 REGION: 1.1647 from Citrus sinensis and AY917195 REGION: 1.1653 from Perilla frutescens.

In some embodiments, the heterologous nucleotide encodes an abietadiene synthase. Illustrative examples of suitable nucleotide sequences include, but are not limited to: (U50768; Abies grandis) and (AY473621; Picea abies).

6.4.2 Recombinant Cells Producing Polyketides

In another aspect, provided herein are methods of detecting polyketide production in a cell or a clonal population of cells, e.g., genetically modified to recombinantly produce one or more polyketide compounds. Polyketide synthesis is mediated by polyketide synthases (PKSs), which are multifunctional enzymes related to fatty acid synthases (FASs). PKSs catalyze the biosynthesis of polyketides through repeated (decarboxylative) Claisen condensations between acylthioesters, usually acetyl, propionyl, malonyl or methylmalonyl. Following each condensation, PKSs introduce structural variability into the polyketide product by catalyzing all, part, or none of a reductive cycle comprising a ketoreduction, dehydration, and enoylreduction on the β-keto group of the growing polyketide chain.

In some embodiments of the methods of detecting a polyketide producing cell provided herein, the polyketide producing cell comprises one or more heterologous nucleotide sequences encoding a PKS system, i.e., one or more PKSs capable of catalyzing the synthesis of a polyketide, to effect increased production of one or more polyketide compounds as compared to a genetically unmodified parent cell.

There are two major classes of polyketide synthases (PKSs): the aromatic PKS and the modular PKS, respectively, which differ in the manner in which the catalytic sites are used. For the aromatic PKS, a minimal system, i.e., the minimal components needed to catalyze the production of a polyketide, comprises a ketosynthase/acyl transferase (KS/AT) catalytic region, a chain length factor (CLF) catalytic region and an acyl carrier protein (ACP) activity. For the modular PKS system, a minimal system comprises a KS catalytic region, an AT catalytic region, and an ACP activity, provided that intermediates in the synthesis are provided as substrates. Where de novo polyketide synthesis is to be required, a minimal modular PKS system further comprises a loading acyl transferase, which includes additional AT and ACP regions.

Thus, in some embodiments, the polyketide producing cell comprises one or more heterologous nucleotide sequences encoding an enzyme comprising a KS catalytic region. In some embodiments, the polyketide producing cell comprises one or more heterologous nucleotide sequences encoding an enzyme comprising an AT catalytic region. In some embodiments, the polyketide producing cell comprises more than one heterologous nucleotide sequence encoding an enzyme comprising an AT catalytic region. In some embodiments, the polyketide producing cell comprises one or more heterologous nucleotide sequences encoding an enzyme comprising a CLF catalytic region. In some embodiments, the polyketide producing cell comprises one or more heterologous nucleotide sequences encoding an enzyme comprising an ACP activity. In some embodiments, the polyketide producing cell comprises more than one heterologous nucleotide sequence encoding an enzyme comprising an ACP activity.

In a particular embodiment, the polyketide producing cell comprises a minimal aromatic PKS system, e.g., heterologous nucleotide sequences encoding an enzyme comprising a KS catalytic region, an enzyme comprising an AT catalytic region, an enzyme comprising a CLF catalytic region, and an enzyme comprising an ACP activity, respectively. In a particular embodiment, the polyketide producing cell comprises a minimal modular PKS system, e.g., heterologous nucleotide sequences encoding an enzyme comprising a KS catalytic region, an enzyme comprising an AT catalytic region, and an enzyme comprising an ACP activity, respectively. In yet another particular embodiment, the polyketide producing cell comprises a modular aromatic PKS system for de novo polyketide synthesis, e.g., heterologous nucleotide sequences encoding an enzyme comprising a KS catalytic region, one or more enzymes comprising an AT catalytic region, and one or more enzymes comprising an ACP activity, respectively.

In some embodiments, the polyketide producing cell comprising a minimal PKS system, e.g., a minimal aromatic PKS system or minimal modular PKS system, as described above, further comprises additional catalytic activities which can contribute to production of the end-product polyketide. In some embodiments, the polyketide producing cell comprises one or more heterologous nucleotide sequences encoding an enzyme comprising a cyclase (CYC) catalytic region, which facilitates the cyclization of the nascent polyketide backbone. In some embodiments, the polyketide producing cell comprises one or more heterologous nucleotide sequences encoding an enzyme comprising a ketoreductase (KR) catalytic region. In some embodiments, the polyketide producing cell comprises one or more heterologous nucleotide sequences encoding an enzyme comprising an aromatase (ARO) catalytic region. In some embodiments, the polyketide producing cell comprises one or more heterologous nucleotide sequences encoding an enzyme comprising an enoylreductase (ER) catalytic region. In some embodiments, the polyketide producing cell comprises one or more heterologous nucleotide sequences encoding an enzyme comprising a thioesterase (TE) catalytic region. In some embodiments, the polyketide producing cell further comprises one or more heterologous nucleotide sequences encoding an enzyme comprising a holo ACP synthase activity, which effects pantetheinylation of the ACP.

In some embodiments, the polyketide producing cell further comprises one or more heterologous nucleotide sequences conferring a postsynthesis polyketide modifying activity. In some embodiments, the polyketide producing cell further comprises one or more heterologous nucleotide sequences encoding an enzyme comprising a glycosylase activity, which effects postsynthesis modifications of polyketides, for example, where polyketides having antibiotic activity are desired. In some embodiments, the polyketide producing cell further comprises one or more heterologous nucleotide sequences encoding an enzyme comprising a hydroxylase activity. In some embodiments, the polyketide producing cell further comprises one or more heterologous nucleotide sequences encoding an enzyme comprising a epoxidase activity. In some embodiments, the polyketide producing cell further comprises one or more heterologous nucleotide sequences encoding an enzyme comprising a methylase activity.

In some embodiments, the polyketide producing cell comprises heterologous nucleotide sequences, for example sequences encoding PKS enzymes and polyketide modification enzymes, capable of producing a polyketide selected from, but not limited to, the following polyketides: Avermectin (see, e.g., U.S. Pat. No. 5,252,474; U.S. Pat. No. 4,703,009; EP Pub. No. 118,367; MacNeil et al., 1993, “Industrial Microorganisms: Basic and Applied Molecular Genetics”; Baltz, Hegeman, & Skatrud, eds. (ASM), pp. 245-256, “A Comparison of the Genes Encoding the Polyketide Synthases for Avermectin, Erythromycin, and Nemadectin”; MacNeil et al., 1992, Gene 115: 119-125; and Ikeda and Omura, 1997, Chem. Res. 97: 2599-2609); Candicidin (FR008) (see, e.g., Hu et al., 1994, Mol. Microbial. 14: 163-172); Carbomycin, Curamycin (see, e.g., Bergh et al., Biotechnol Appl Biochem. 1992 February; 15(1):80-9); Daunorubicin (see, e.g., J Bacteriol. 1994 October; 176(20):6270-80); Epothilone (see, e.g., PCT Pub. No. 99/66028; and PCT Pub. No. 00/031247); Erythromycin (see, e.g., PCT Pub. No. 93/13663; U.S. Pat. No. 6,004,787; U.S. Pat. No. 5,824,513; Donadio et al., 1991, Science 252:675-9; and Cortes et al., Nov. 8, 1990, Nature 348:176-8); FK-506 (see, e.g., Motamedi et al., 1998; Eur. J. Biochem. 256: 528-534; and Motamedi et al., 1997, Eur. J Biochem. 244: 74-80); FK-520 (see, e.g., PCT Pub. No. 00/020601; and Nielsen et al., 1991, Biochem. 30:5789-96); Griseusin (see, e.g., Yu et al., J Bacteriol. 1994 May; 176(9):2627-34); Lovastatin (see, e.g., U.S. Pat. No. 5,744,350); Frenolycin (see, e.g., Khosla et al., Bacteriol. 1993 April; 175(8):2197-204; and Bibb et al., Gene 1994 May 3; 142(1):31-9); Granaticin (see, e.g., Sherman et al., EMBO J. 1989 September; 8(9):2717-25; and Bechtold et al., Mol Gen Genet. 1995 Sep. 20; 248(5):610-20); Medermycin (see, e.g., Ichinose et al., Microbiology 2003 July; 149(Pt 7):1633-45); Monensin (see, e.g., Arrowsmith et al., Mol Gen Genet. 1992 August; 234(2):254-64); Nonactin (see, e.g., FEMS Microbiol Lett. 2000 Feb. 1; 183(1):171-5); Nanaomycin (see, e.g., Kitao et al., J Antibiot (Tokyo). 1980 July; 33(7):711-6); Nemadectin (see, e.g., MacNeil et al., 1993, supra); Niddamycin (see, e.g., PCT Pub. No. 98/51695; and Kakavas et al., 1997, J. Bacteriol. 179: 7515-7522); Oleandomycin (see e.g., Swan et al., 1994, Mol. Gen. Genet. 242: 358-362; PCT Pub. No. 00/026349; Olano et al., 1998, Mol. Gen. Genet. 259(3): 299-308; and PCT Pat. App. Pub. No. WO 99/05283); Oxytetracycline (see, e.g., Kim et al., Gene. 1994 Apr. 8; 141(1):141-2); Picromycin (see, e.g., PCT Pub. No. 99/61599; PCT Pub. No. 00/00620; Xue et al., 1998, Chemistry & Biology 5(11): 661-667; Xue et al., October 1998, Proc. Natl. Acad. Sci. USA 95: 12111 12116); Platenolide (see, e.g., EP Pub. No. 791,656; and U.S. Pat. No. 5,945,320); Rapamycin (see, e.g., Schwecke et al., August 1995, Proc. Natl. Acad. Sci. USA 92:7839-7843; and Aparicio et al., 1996, Gene 169: 9-16); Rifamycin (see, e.g., PCT Pub. No. WO 98/07868; and August et al., Feb. 13, 1998, Chemistry & Biology, 5(2): 69-79); Sorangium (see, e.g., U.S. Pat. No. 6,090,601); Soraphen (see, e.g., U.S. Pat. No. 5,716,849; Schupp et al., 1995, J. Bacteriology 177: 3673-3679); Spinocyn (see, e.g., PCT Pub. No. 99/46387); Spiramycin (see, e.g., U.S. Pat. No. 5,098,837); Tetracenomycin (see, e.g., Summers et al., J Bacteriol. 1992 March; 174(6):1810-20; and Shen et al., J Bacteriol. 1992 June; 174(11):3818-21); Tetracycline (see, e.g., J Am Chem Soc. 2009 Dec. 9; 131(48):17677-89); Tylosin (see, e.g., U.S. Pat. No. 5,876,991; U.S. Pat. No. 5,672,497; U.S. Pat. No. 5,149,638; EP Pub. No. 791,655; EP Pub. No. 238,323; Kuhstoss et al., 1996, Gene 183:231-6; and Merson-Davies and Cundliffe, 1994, Mol. Microbiol. 13: 349-355); and 6-methylsalicyclic acid (see, e.g., Richardson et al., Metab Eng. 1999 April; 1(2):180-7; and Shao et al., Biochem Biophys Res Commun. 2006 Jun. 23; 345(1):133-9).

6.4.3 Recombinant Cells Producing Fatty Acids

In another aspect, provided herein are methods of detecting fatty acid production in a cell or a clonal population of cells, e.g., genetically modified to recombinantly produce one or more fatty acids. Fatty acid synthesis is mediated by fatty acid synthases (FAS), which catalyze the initiation and elongation of acyl chains. The acyl carrier protein (ACP) along with the enzymes in the FAS pathway control the length, degree of saturation, and branching of the fatty acid produced. The fatty acid biosynthetic pathway involves the precursors acetyl-CoA and malonyl-CoA. The steps in this pathway are catalyzed by enzymes of the fatty acid biosynthesis (fab) and acetyl-CoA carboxylase (ace) gene.

In some embodiments of the methods of detecting a fatty acid producing cell provided herein, the fatty acid producing cell comprises one or more heterologous nucleotide sequences encoding acetyl-CoA synthase and/or malonyl-CoA synthase, to effect increased production of one or more fatty acids as compared to a genetically unmodified parent cell.

For example, to increase acetyl-CoA production, one or more of the following genes can be expressed in the cell: pdh, panK, aceEF (encoding the EIp dehydrogenase component and the E2p dihydrolipoamide acyltransferase component of the pyruvate and 2-oxoglutarate dehydrogenase complexes), fabH, fabD, fabG, acpP, and fabF. Illustrative examples of nucleotide sequences encoding such enzymes include, but are not limited to: pdh (BAB34380, AAC73227, AAC73226), panK (also known as coaA, AAC76952), aceEF (AAC73227, AAC73226), fabH (AAC74175), fabD (AAC74176), fabG (AAC74177), acpP (AAC74178), fabF (AAC74179).

In some embodiments, increased fatty acid levels can be effected in the cell by attenuating or knocking out genes encoding proteins involved in fatty acid degradation. For example, the expression levels of fadE, gpsA, idhA, pflb, adhE, pta, poxB, ackA, and/or ackB can be attenuated or knocked-out in an engineered host cell using techniques known in the art. Illustrative examples of nucleotide sequences encoding such proteins include, but are not limited to: fadE (AAC73325), gspA (AAC76632), IdhA (AAC74462), pflb (AAC73989), adhE (AAC74323), pta (AAC75357), poxB (AAC73958), ackA (AAC75356), and ackB (BAB81430). The resulting host cells will have increased acetyl-CoA production levels when grown in an appropriate environment.

In some embodiments, the fatty acid producing cell comprises a heterologous nucleotide sequence encoding an enzyme that can convert acetyl-CoA into malonyl-CoA, e.g., the multisubunit AccABCD protein. An illustrative example of a suitable nucleotide sequence encoding AccABCD includes but is not limited to accession number AAC73296, EC 6.4.1.2.

In some embodiments, the fatty acid producing cell comprises a heterologous nucleotide sequence encoding a lipase. Illustrative examples of suitable nucleotide sequences encoding a lipase include, but are not limited to accession numbers CAA89087 and CAA98876.

In some embodiments, increased fatty acid levels can be effected in the cell by inhibiting PlsB, which can lead to an increase in the levels of long chain acyl-ACP, which will inhibit early steps in the fatty acid biosynthesis pathway (e.g., accABCD, fabH, and fabl). The expression level of PlsB can be attenuated or knocked-out in an engineered host cell using techniques known in the art. An illustrative example of a suitable nucleotide sequence encoding PlsB includes but is not limited to accession number AAC77011. In particular embodiments, the plsB D31 IE mutation can be used to increase the amount of available acyl-CoA in the cell.

In some embodiments, increased production of monounsaturated fatty acids can be effected in the cell by overexpressing an sfa gene, which would result in suppression of fabA. An illustrative example of a suitable nucleotide sequence encoding sfa includes but is not limited to accession number AAN79592.

In some embodiments, increased fatty acid levels can be effected in the cell by modulating the expression of an enzyme which controls the chain length of a fatty acid substrate, e.g., a thioesterase. In some embodiments, the fatty acid producing cell has been modified to overexpress a tes or fat gene. Illustrative examples of suitable tes nucleotide sequences include but are not limited to accession numbers: (tesA: AAC73596, from E. Coli, capable of producing C_{18:1} fatty acids) and (tesB: AAC73555 from E. Coli). Illustrative examples of suitable fat nucleotide sequences include but are not limited to: (fatB: Q41635 and AAA34215, from Umbellularia california, capable of producing C_{12:0} fatty acids), (fatB2: Q39513 and AAC49269, from Cuphea hookeriana, capable of producing C_{8:0}-C_{10:0} fatty acids), (fatB3: AAC49269 and AAC72881, from Cuphea hookeriana, capable of producing C_{14:0}-C_{16:0} fatty acids), (fatB: Q39473 and AAC49151, from Cinnamonum camphorum, capable of producing C_{14:0} fatty acids), (fatB [M141T]: CAA85388, from mArabidopsis thaliana, capable of producing C_{16:1} fatty acids), (fatA: NP 189147 and NP 193041, from Arabidopsis thaliana, capable of producing C_{18:1} fatty acids), (fatA: CAC39106, from Bradvrhiizobium japonicum, capable of preferentially producing C_{18:1} fatty acids), (fatA: AAC72883, from Cuphea hookeriana, capable of producing C_{18:1} fatty acids), and (fatA1, AAL79361 from Helianthus annus).

In some embodiments, increased levels of C₁₀ fatty acids can be effected in the cell by attenuating the expression or activity of thioesterase C₁₈ using techniques known in the art. Illustrative examples of suitable nucleotide sequences encoding thioesterase C₁₈ include, but are not limited to accession numbers AAC73596 and P0ADA1. In other embodiments, increased levels of C₁₀ fatty acids can be effected in the cell by increasing the expression or activity of thioesterase C₁₀ using techniques known in the art. An illustrative example of a suitable nucleotide sequence encoding thioesterase C₁₀ includes, but is not limited to accession number Q39513.

In some embodiments, increased levels of C₁₄ fatty acids can be effected in the cell by attenuating the expression or activity of endogenous thioesterases that produce non-C₁₄ fatty acids, using techniques known in the art. In other embodiments, increased levels of C₁₄ fatty acids can be effected in the cell by increasing the expression or activity of thioesterases that use the substrate C₁₄-ACP, using techniques known in the art. An illustrative example of a suitable nucleotide sequence encoding such a thioesterase includes, but is not limited to accession number Q39473.

In some embodiments, increased levels of C₁₂ fatty acids can be effected in the cell by attenuating the expression or activity of endogenous thioesterases that produce non-C₁₂ fatty acids, using techniques known in the art. In other embodiments, increased levels of C₁₂ fatty acids can be effected in the cell by increasing the expression or activity of thioesterases that use the substrate C₁₂-ACP, using techniques known in the art. An illustrative example of a suitable nucleotide sequence encoding such a thioesterase includes, but is not limited to accession number Q41635.

6.4.4 Additional Genetic Modifications

In some embodiments of the methods and compositions provided herein, the genetically modified cell engineered to produce one or more water-immiscible compounds further comprises one or more genetic modifications which confer to the cell useful properties in the context of industrial fermentation.

In some embodiments, the cell further comprises one or more heterologous nucleotide sequences encoding one or more proteins that increase flocculation. Flocculation is the asexual, reversible, and calcium-dependent aggregation of microbial cells to form flocs containing large numbers of cells that rapidly sediment to the bottom of the liquid growth substrate. Flocculation is of significance in industrial fermentations of yeast, e.g., for the production of bioethanol, wine, beer, and other products, because it greatly simplifies the processes for separating the suspended yeast cells from the fermentation products produced therefrom in the industrial fermentation. The separation may be achieved by centrifugation or filtration, but separation by these methods is time-consuming and expensive. Clarification can be alternatively achieved by natural settling of the microbial cells. Although single microbial cells tend to settle over time, natural settling becomes a viable option in industrial processes only when cells aggregate (i.e., flocculate). Recent studies demonstrate that the flocculation behavior of yeast cells can be tightly controlled and fine-tuned to satisfy specific industrial requirements (see, e.g., Governder et al., Appl Environ Microbiol. 74(19):6041-52 (2008), the contents of which are hereby incorporated by reference in their entirety). Flocculation behavior of yeast cells is dependent on the function of specific flocculation proteins, including, but not limited to, products of the FLO1, FLO5, FLO8, FLO9, FLO10, and FLO11 genes. Thus, in some embodiments, the genetically modified cell engineered to produce one or more water-immiscible compounds described herein comprises one or more heterologous nucleotide sequences encoding one or more flocculation proteins selected from the group consisting of Flo1p, Flo5p, Flo8p, Flo9p, Flo10p, and Flo11p.

In some embodiments, the cell is sporulation impaired and/or endogenous mating impaired. A sporulation and/or endogenous mating impaired genetically modified microbial cell poses reduced risk of: (1) dissemination in nature; and (2) exchange of genetic material between the genetically modified microbial cell and a wild-type microbe that is not compromised in its ability to disseminate in nature. In yeast, the ability of diploid microbial cells to sporulate, and of haploid microbial cells to mate, is dependent on the function of specific gene products. Among these in yeast are products of sporulation genes, such as of the IME1, IME2, NDT80, SPO11, SPO20, AMA1, HOP2, and SPO21 genes, and products of pheromone response genes, such as of the STE5, STE4, STEI8, STE12, STE7 and STE11 genes.

In some embodiments, the cell is a haploid yeast cell in which one or more of the following pheromone response genes is functionally disrupted: STE5, STE4, STE18, STE12, STE7, and STE11. In some embodiments, the cell is a haploid yeast cell in which one or more of the following sporulation genes is functionally disrupted: IME1, IME2, NDT80, SPO11, SPO20, AMA1, HOP2, and SPO21. In some embodiments, the cell is a haploid yeast cell in which one or more of the following pheromone response genes: STE5, STE4, STE18, STE12, STE7, and STE11, and one or more of the following sporulation genes: IME1, IME2, NDT80, SPO11, SPO20, AMA1, HOP2, and SPO21, are functionally disrupted. In some embodiments, the cell is a haploid yeast cell in which the IME1 gene and the STE5 gene are functionally disrupted. In some embodiments, the cell is a haploid yeast cell in which the IME1 gene and the STE5 gene are functionally disrupted and that comprises a heterologous nucleotide sequence encoding an enzyme that can convert HMG-CoA into mevalonate. In some embodiments, the cell is a haploid yeast cell in which the IME1 gene and the STE5 gene are functionally disrupted, and that comprises a heterologous nucleotide sequence encoding an enzyme that can convert mevalonate into mevalonate 5-phosphate.

In some embodiments, the cell is a diploid yeast cell in which both copies of one or more of the following pheromone response genes are functionally disrupted: STE5, STE4, STE18, STE12, STE7, and STE11. In some embodiments, the cell is a diploid yeast cell in which both copies of one or more of the following sporulation genes are functionally disrupted: IME1, IME2, NDT80, SPO11, SPO20, AMA1, HOP2, and SPO21. In some embodiments, the cell is a diploid yeast cell in which both copies of one or more of the following pheromone response genes: STE5, STE4, STE18, STE12, STE7, and STE11, and both copies of one or more of the following sporulation genes: IME1, IME2, NDT80, SPO11, SPO20, AMA1, HOP2, and SPO21, are functionally disrupted. In some embodiments, the cell is a diploid yeast cell in which both copies of the IME1 gene and both copies of the STE5 gene are functionally disrupted. In some embodiments, the cell is a diploid yeast cell in which both copies of the IME1 gene and both copies of the STE5 gene are functionally disrupted, and that comprises a heterologous nucleotide sequence encoding an enzyme that can convert HMG-CoA into mevalonate. In some embodiments, the cell is a diploid yeast cell in which both copies of the IME1 gene and both copies of the STE5 gene are functionally disrupted, and that comprises a heterologous nucleotide sequence encoding an enzyme that can convert mevalonate into mevalonate 5-phosphate.

Methods and compositions useful for the introduction of heterologous sequences encoding flocculation proteins, and for the functional disruption of one or more sporulation genes and/or pheromone response genes, are described in U.S. Patent Application Publication No. 2010/0304490 and U.S. Patent Application Publication No. 2010/0311065, the disclosures of which are hereby incorporated by reference in their entireties.

In some embodiments, the cell comprises a functional disruption in one or more biosynthesis genes, wherein said cell is auxotrophic as a result of said disruption. In certain embodiments, the cell does not comprise a heterologous nucleotide sequence that confers resistance to an antibiotic compound. In other embodiments, the cell comprises one or more selectable marker genes. In some embodiments, the selectable marker is an antibiotic resistance marker. Illustrative examples of antibiotic resistance markers include, but are not limited to the BLA, NAT1, PAT, AUR1-C, PDR4, SMR1, CAT, mouse dhfr, HPH, DSDA, KAN^{R}, and SH BLE gene products. The BLA gene product from E. coli confers resistance to beta-lactam antibiotics (e.g., narrow-spectrum cephalosporins, cephamycins, and carbapenems (ertapenem), cefamandole, and cefoperazone) and to all the anti-gram-negative-bacterium penicillins except temocillin; the NAT1 gene product from S. noursei confers resistance to nourseothricin; the PAT gene product from S. viridochromogenes Tu94 confers resistance to bialophos; the AUR1-C gene product from Saccharomyces cerevisiae confers resistance to Auerobasidin A (AbA); the PDR4 gene product confers resistance to cerulenin; the SMR1 gene product confers resistance to sulfometuron methyl; the CAT gene product from Tn9 transposon confers resistance to chloramphenicol; the mouse dhfr gene product confers resistance to methotrexate; the HPH gene product of Klebsiella pneumonia confers resistance to Hygromycin B; the DSDA gene product of E. coli allows cells to grow on plates with D-serine as the sole nitrogen source; the KAN^{R} gene of the Tn903 transposon confers resistance to G418; and the SH BLE gene product from Streptoalloteichus hindustanus confers resistance to Zeocin (bleomycin). In some embodiments, the antibiotic resistance marker is excised, e.g., from the host cell genome after the cell has been genetically modified to effect increased water-immiscible compound production. Methods and compositions useful for the precise excision of nucleotide sequences, e.g., sequences encoding such antibiotic resistance markers from the genome of a genetically modified host cell, are described in U.S. patent application Ser. No. 12/978,061, filed on Dec. 23, 2010, the disclosure of which is incorporated herein by reference in its entirety.

In some embodiments, the selectable marker rescues an auxotrophy (e.g., a nutritional auxotrophy) in the genetically modified microbial cell. In such embodiments, a parent microbial cell comprises a functional disruption in one or more gene products that function in an amino acid or nucleotide biosynthetic pathway, such as, for example, the HIS3, LEU2, LYS1, LYS2, MET15, TRP1, ADE2, and URA3 gene products in yeast, which renders the parent microbial cell incapable of growing in media without supplementation with one or more nutrients (auxotrophic phenotype). The auxotrophic phenotype can then be rescued by transforming the parent microbial cell with an expression vector or chromosomal integration encoding a functional copy of the disrupted gene product, and the genetically modified microbial cell generated can be selected for based on the loss of the auxotrophic phenotype of the parent microbial cell. Utilization of the URA3, TRP1, and LYS2 genes as selectable markers has a marked advantage because both positive and negative selections are possible. Positive selection is carried out by auxotrophic complementation of the URA3, TRP1, and LYS2 mutations, whereas negative selection is based on specific inhibitors, i.e., 5-fluoro-orotic acid (FOA), 5-fluoroanthranilic acid, and a-aminoadipic acid (aAA), respectively, that prevent growth of the prototrophic strains but allows growth of the URA3, TRP1, and LYS2 mutants, respectively.

In other embodiments, the selectable marker rescues other non-lethal deficiencies or phenotypes that can be identified by a known selection method.

Methods for genetically modifying microbes using expression vectors or chromosomal integration constructs, e.g., to effect increased production of one or more water-immiscible compounds in a host cell, or to confer useful properties to such cells as described above, are well known in the art. See, for example, Sherman, F., et al., Methods Yeast Genetics, Cold Spring Harbor Laboratory, N.Y. (1978); Guthrie, C., et al. (Eds.) Guide To Yeast Genetics and Molecular Biology Vol. 194, Academic Press, San Diego (1991); Sambrook et al., 2001, Molecular Cloning—A Laboratory Manual, 3^{rd} edition, Cold Spring Harbor Laboratory, Cold Spring Harbor, N.Y.; and Ausubel et al., eds., Current Edition, Current Protocols in Molecular Biology, Greene Publishing Associates and Wiley Interscience, NY.; the disclosures of which are incorporated herein by reference. In addition, inhibition of gene expression, e.g., which results in increased production of one or more water-immiscible compounds in the cell, may be accomplished by deletion, mutation, and/or gene rearrangement. It can also be carried out with the use of antisense RNA, siRNA, miRNA, ribozymes, triple stranded DNA, and transcription and/or translation inhibitors. In addition, transposons can be employed to disrupt gene expression, for example, by inserting it between the promoter and the coding region, or between two adjacent genes to inactivate one or both genes.

In some embodiments, increased production of water-immiscible compound in the cell is effected by the use of expression vectors to express a particular protein, e.g., a protein involved in a biosynthetic pathway as described above. Generally, expression vectors are recombinant polynucleotide molecules comprising replication signals and expression control sequences, e.g., promoters and terminators, operatively linked to a nucleotide sequence encoding a polypeptide. Expression vectors useful for expressing polypeptide-encoding nucleotide sequences include viral vectors (e.g., retroviruses, adenoviruses and adenoassociated viruses), plasmid vectors, and cosmids. Illustrative examples of expression vectors suitable for use in yeast cells include, but are not limited to CEN/ARS and 2μ plasmids. Illustrative examples of promoters suitable for use in yeast cells include, but are not limited to the promoter of the TEF1 gene of K. lactis, the promoter of the PGK1 gene of Saccharomyces cerevisiae, the promoter of the TDH3 gene of Saccharomyces cerevisiae, repressible promoters, e.g., the promoter of the CTR3 gene of Saccharomyces cerevisiae, and inducible promoters, e.g., galactose inducible promoters of Saccharomyces cerevisiae (e.g., promoters of the GAL1, GAL7, and GAL10 genes). Expression vectors and chromosomal integration constructs can be introduced into microbial cells by any method known to one of skill in the art without limitation. See, for example, Hinnen et al., Proc. Natl. Acad. Sci. USA 75:1292-3 (1978); Cregg et al., Mol. Cell. Biol. 5:3376-3385 (1985); U.S. Pat. No. 5,272,065; Goeddel et al., eds, 1990, Methods in Enzymology, vol. 185, Academic Press, Inc., CA; Krieger, 1990, Gene Transfer and Expression—A Laboratory Manual, Stockton Press, NY; Sambrook et al., 1989, Molecular Cloning—A Laboratory Manual, Cold Spring Harbor Laboratory, NY; and Ausubel et al., eds., Current Edition, Current Protocols in Molecular Biology, Greene Publishing Associates and Wiley Interscience, NY. Exemplary techniques include, but are not limited to, spheroplasting, electroporation, PEG 1000 mediated transformation, and lithium acetate or lithium chloride mediated transformation.

### 7. EXAMPLES

### 7.1 Example 1

### Generation of Genetically Modified Haploid Cells

This example describes the generation of genetically modified haploid S. cerevisiae cells engineered to produce isoprenoid.

The Phase I integration construct comprises as an integrating sequence nucleotide sequences that encode a selectable marker (hygA, which confers resistance to hygromycin B); two enzymes of the S. cerevisiae MEV pathway (the truncated HMG1 coding sequence, which encodes a truncated HMG-CoA reductase, and the ERG13 coding sequence, which encodes HMG-CoA synthase), and another enzyme of S. cerevisiae (the ERG10 coding sequence, which encodes acetoacetyl-CoA thiolase), under control of galactose-inducible promoters (promoters of the S. cerevisiae genes GAL1 and GAL10); flanked by homologous sequences consisting of upstream and downstream nucleotide sequences of the S. cerevisiae GAL80 locus. Upon introduction into a S. cerevisiae host cell, the Phase I integration construct can integrate by homologous recombination into the GAL80 locus of the S. cerevisiae host cell genome, and functionally disrupt the GAL80 locus by replacing the GAL80 coding sequence with its integrating sequence. The Phase I integration construct was cloned into the TOPO Zero Blunt II cloning vector (Invitrogen, Carlsbad, Calif.), yielding plasmid TOPO-Phase I integration construct. The construct was propagated in TOP10 cells grown on LB agar containing 50 μg/ml kanamycin.

The Phase II integration construct comprises as an integrating sequence nucleotide sequences encoding a selectable marker (natA, which confers resistance to nourseothricin) and several enzymes of the S. cerevisiae MEV pathway (the ERG12 coding sequence, which encodes mevalonate kinase, and the ERG8 coding sequence, which encodes phosphomevalonate kinase), under control of galactose-inducible promoters (promoters of the S. cerevisiae genes GAL1 and GAL10); as well as the coding sequence of the S. cerevisiae GAL4 gene under control of the GAL4oc promoter (GAL4 promoter comprising a mutation that removes the MIG1 binding site thus making the promoter less sensitive to the repression by glucose); flanked by homologous sequences consisting of upstream and downstream nucleotide sequences of the S. cerevisiae LEU2 locus. Upon introduction into a S. cerevisiae host cell, the Phase II integration construct can integrate by homologous recombination into the LEU2 locus of the S. cerevisiae host cell genome, and functionally disrupt the LEU2 locus by replacing the LEU2 coding sequence with its integrating sequence. The Phase II integration construct was cloned into the TOPO Zero Blunt II cloning vector, yielding plasmid TOPO-Phase II integration construct. The construct was propagated in TOP10 cells (Invitrogen, Carlsbad, Calif.) grown on LB agar containing 50 μg/ml kanamycin.

The Phase III integration construct comprises as an integrating sequence nucleotide sequences encoding a selectable marker (kanA, which confers resistance to G418); an enzyme of the S. cerevisiae MEV pathway (the ERG19 coding sequence, which encodes diphosphomevalonate decarboxylase), and two enzymes of S. cerevisiae involved in converting the product of the MEV pathway, IPP, into FPP (the ERG20 coding sequence, which encodes farnesyl pyrophosphate synthase, and the IDI1 coding sequence, which encodes isopentenyl pyrophosphate decarboxylase), under control of galactose-inducible promoters (promoters of the S. cerevisiae genes GAL1, GAL10, and GAL7); as well as the promoter of the S. cerevisiae CTR3 gene; flanked by upstream and coding nucleotide sequences of the S. cerevisiae ERG9 locus. Upon introduction into a S. cerevisiae host cell, the Phase II integration construct can integrate by homologous recombination upstream of the ERG9 locus of the S. cerevisiae host cell genome, replacing the native ERG9 promoter with the CTR3 promoter in such a way that the expression of ERG9 (squalene synthase) can be modulated by copper. The Phase III integration construct was cloned into the TOPO Zero Blunt II cloning vector, yielding plasmid TOPO-Phase III integration construct. The construct was propagated in TOP10 cells grown on LB agar containing 50 μg/ml kanamycin.

The Phase I marker recycling construct comprises nucleotide sequences encoding a selectable marker (URA3, which confers the ability to grow on media lacking uracil); and an enzyme of A. annua (the FS coding sequence, which encodes farnesene synthase), under regulatory control of the promoter of the S. cerevisiae GAL7 gene; flanked by upstream nucleotide sequences of the S. cerevisiae GAL80 locus and coding sequences of the S. cerevisiae HMG1 gene. Upon introduction into a S. cerevisiae host cell, the Phase I marker recycling construct can integrate by homologous recombination into the already integrated Phase I integrating sequence such that the selective marker hphA is replaced with URA3.

The Phase II marker recycling construct comprises nucleotide sequences encoding a selectable marker (URA3, which confers ability to grow on media lacking uracil) and an enzyme of A annua (the FS coding sequence, which encodes farnesene synthase), under regulatory control of the promoter of the S. cerevisiae GAL7 gene; flanked by upstream nucleotide sequences of the S. cerevisiae LEU2 locus and coding sequences of the S. cerevisiae ERG12 gene. Upon introduction into a S. cerevisiae host cell, the Phase II marker recycling construct can integrate by homologous recombination into the already integrated Phase II integrating sequence such that the selective marker natA is replaced with URA3.

The Phase III marker recycling construct comprises nucleotide sequences encoding a selectable marker (URA3, which confers the ability to grow on media lacking uracil) and an enzyme of A annua the FS coding sequence encodes farnesene synthase), under regulatory control of the promoter of the S. cerevisiae GAL7 gene; flanked by upstream nucleotide sequences of the S. cerevisiae ERG9 locus and coding sequences of the S. cerevisiae ERG19 gene. Upon introduction into a S. cerevisiae host cell, the Phase II marker recycling construct can integrate by homologous recombination into the already integrated Phase III integrating sequence such that the selective marker kanA is replaced with URA3.

Expression plasmid pAM404 encodes a β-farnesene synthase. The nucleotide sequence insert was generated synthetically, using as a template the coding sequence of the β-farnesene synthase gene of Artemisia annua (GenBank accession number AY835398) codon-optimized for expression in Saccharomyces cerevisiae.

Starter host strain Y1198 was generated by resuspending active dry PE-2 yeast (isolated in 1994; gift from Santelisa Vale, Sertãozinho, Brazil) in 5 mL of YPD medium containing 100 ug/mL carbamicillin and 50 ug/mL kanamycin. The culture was incubated overnight at 30° C. on a rotary shaker at 200 rpm. An aliquot of 10 uL of the culture was then plated on a YPD plate and allowed to dry. The cells were serially streaked for single colonies, and incubated for 2 days at 30° C. Twelve single colonies were picked, patched out on a new YPD plate, and allowed to grow overnight at 30° C. The strain identities of the colonies were verified by analyzing their chromosomal sizes on a Bio-Rad CHEF DR H system (Bio-Rad, Hercules, Calif.) using the Bio-Rad CHEF Genomic DNA Plug Kit (Bio-Rad, Hercules, Calif.) according to the manufacturer's specifications. One colony was picked and stocked as strain Y1198.

Strains Y1661, Y1662, Y1663, and Y1664 were generated from strain Y1198 by rendering the strain haploid to permit genetic engineering. Strain Y1198 was grown overnight in 5 mL of YPD medium at 30° C. in a glass tube in a roller drum. The OD₆₀₀ was measured, and the cells were diluted to an OD₆₀₀ of 0.2 in 5 mL of YP medium containing 2% potassium acetate. The culture was grown overnight at 30° C. in a glass tube in a roller drum. The OD₆₀₀ was measured again, and 4 OD₆₀₀*mL of cells was collected by centrifugation at 5,000×g for 2 minutes. The cell pellet was washed once with sterile water, and then resuspended in 3 mL of 2% potassium acetate containing 0.02% raffinose. The cells were grown for 3 days at 30° C. in a glass tube in a roller drum. Sporulation was confirmed by microscopy. An aliquot of 33 μL of the culture was transferred to a 1.5 mL microfuge tube and was centrifuged at 14,000 rpm for 2 minutes. The cell pellet was resuspended in 50 μL of sterile water containing 2 μL of 10 mg/mL Zymolyase 100T (MP Biomedicals, Solon, Ohio), and the cells were incubated for 10 minutes in a 30° C. waterbath. The tube was transferred to ice, and 150 μL of ice cold water was added. An aliquot of 10 μL of this mixture was added to a 12 mL YPD plate, and tetrads were dissected on a Singer MSM 300 dissection microscope (Singer, Somerset, UK). The YPD plate was incubated at 30° C. for 3 days, after which spores were patched out onto a fresh YPD plate and grown overnight at 30° C. The mating types of each spore from 8 four-spore tetrads were analyzed by colony PCR. A single 4 spore tetrad with 2 MATa and 2 MATα spores was picked and stocked as strains Y1661 (MATa), Y1662 (MATa), Y1663 (MATα), and Y1664 (MATα).

For yeast cell transformations, 25 ml of Yeast Extract Peptone Dextrose (YPD) medium was inoculated with a single colony of a starting host strain. The culture was grown overnight at 30° C. on a rotary shaker at 200 rpm. The OD₆₀₀ of the culture was measured, and the culture was then used to inoculate 50 ml of YPD medium to an OD₆₀₀ of 0.15. The newly inoculated culture was grown at 30° C. on a rotary shaker at 200 rpm up to an OD₆₀₀ of 0.7 to 0.9, at which point the cells were transformed with 1 μg of DNA. The cells were allowed to recover in YPD medium for 4 hours before they were plated on agar containing a selective agent to identify the host cell transformants.

Host strain Y1515 was generated by transforming strain Y1664 with plasmid TOPO-Phase I integration construct digested to completion using PmeI restriction endonuclease. Host cell transformants were selected on YPD medium containing 300 ug/mL hygromycin B, and positive transformants comprising the Phase I integrating sequence integrated at the GAL80 locus were verified by the PCR amplification.

Host strain Y1762 was generated by transforming strain Y1515 with plasmid TOPO-Phase II integration construct digested to completion using PmeI restriction endonuclease. Host cell transformants were selected on YPD medium containing 100 ug/mL nourseothricin, and positive transformants comprising the Phase II integrating sequence integrated at the LEU2 locus were verified by the PCR amplification.

Host strain Y1770 was generated by transforming strain Y1762 in two steps with expression plasmid pAM404 and plasmid TOPO-Phase III integration construct digested to completion using PmeI restriction endonuclease. Host cell transformants with pAM404 were selected on Complete Synthetic Medium (CSM) lacking methionine and leucine. Host cell transformants with pAM404 and Phase III integration construct were selected on CSM lacking methionine and leucine and containing 200 ug/mL G418 (Geneticin®), and positive transformants comprising the Phase III integrating sequence integrated at the ERG9 locus were verified by the PCR amplification.

Host strain Y1793 was generated by transforming strain Y1770 with a URA3 knockout construct. The URA3 knockout construct comprises upstream and downstream sequences of the URA3 locus (generated from Saccharomyces cerevisiae strain CEN.PK2 genomic DNA). Host cell transformants were selected on YPD medium containing 5-FOA.

Host strain YAAA was generated by transforming strain Y1793 with the Phase I marker recycling construct. Host cell transformants were selected on CSM lacking methionine and uracil. The URA3 marker was excised by growing the cells overnight in YPD medium at 30° C. on a rotary shaker at 200 rpm, and then plating the cells onto agar containing 5-FOA. Marker excision was confirmed by colony PCR.

Host strain YBBB was generated by transforming strain YAAA with the Phase II marker recycling construct. Host cell transformants were selected on CSM lacking methionine and uracil. The URA3 marker was excised by growing the cells overnight in YPD medium at 30° C. on a rotary shaker at 200 rpm, and then plating the cells onto agar containing 5-FOA. Marker excision was confirmed by colony PCR.

Host strain Y1912 was generated by transforming strain YBBB with the Phase III marker recycling construct. Host cell transformants were selected on CSM lacking methionine and uracil. The URA3 marker was excised by growing the cells overnight in YPD medium at 30° C. on a rotary shaker at 200 rpm, and then plating the cells onto agar containing 5-FOA. Marker excision was confirmed by colony PCR.

### 7.2 Example 2

### Determination of Spectral Conditions for Specifically Detecting Recombinantly Produced Water Immiscible Compound (WIC)

This example provides an exemplary method for determining spectral conditions useful for the specific detection of farnesene produced by a population of recombinant yeast cells, prepared as described in Example 1, using the lipophilic dye Nile Red. As demonstrated below, these spectral conditions enable the detection of farnesene-specific fluorescence emitted by Nile Red, with little to no spillover of cellular membrane-specific (i.e., biomass-specific) fluorescence, thus allowing for an evaluation of farnesene production that is uninfluenced by biomass. A biomass-independent assessment of recombinant compound production is critical when comparing pluralities of cell populations, for example, when screening libraries of recombinant producers, where cell viability and biomass can be negatively impacted by production of the recombinant product.

Nile Red is a lipid-soluble fluorescent dye that has frequently been used to evaluate the lipid content of animal cells and microorganisms, including mammalian cells, bacteria, yeasts and microalgae. These studies by in large have focused on the detection of natively produced intracellular lipids under spectral conditions based largely on the excitation and emission maxima of known nonpolar solvents or neutral lipids. Greenspan et al. (J. Cell Biology 100:965-973 (1985)) reported that selectivity for cytoplasmic lipid droplets was obtained when the cells were viewed for yellow-gold fluorescence, i.e., excitation wavelengths of 450-500 nm and emission wavelengths of >528 nm. While these spectral conditions were purportedly sufficient to distinguish neutral lipid droplets from cellular membranes within single cells viewed by light microscopy or flow cytometry, no evaluation was made of the amount of yellow-gold fluorescence contributed by cellular membranes in cell populations of varying optical densities (ODs), particularly by spectrophotometric detection. In addition, no evaluation was made of the ability of Nile Red to detect lipids or other neutral compounds that were secreted or diffused into extracellular solution.

To determine the biomass-specific contribution to the yellow-gold fluorescence of farnesene in the presence of cell populations of varying ODs, a cell/farnesene titration matrix was prepared and stained with Nile Red, and fluorescence in the yellow-gold spectrum was detected. As depicted in FIG. 1, populations of naïve yeast cells of OD 5, 10, 15, 20 and 25, and a no-cell control were plated in growth medium along the x-axis of a 96-well microtiter plate, while increasing concentrations of purified farnesene (0, 2, 4, 6, 8 and 10 g/L) were added to wells along the y-axis. 2 μL of a 100 μg/ml solution of Nile Red in DMSO were added to 98 μL of solution comprising cells and/or farnesene. The matrix was viewed under two different spectral conditions within the yellow-gold spectrum: (1) an excitation wavelength of 488 nm and an emission wavelength of 515 nm (FIG. 1); and (2) an excitation wavelength of 500 nm and an emission wavelength of 550 nm (FIG. 2).

As shown in FIG. 1, when viewed at 488ₑₓ/515ₑₘ, fluorescence is highly influenced by both increasing cell density and increasing farnesene. While fluorescence increases with increasing farnesene concentration along the y-axis, fluorescence also increases along the x-axis with increasing cell density. In particular, the difference in fluorescence between OD 5 to OD 25 in the absence of farnesene was greater than 3-fold. Similar results were observed at 500ₑₓ/550ₑₘ (FIG. 2A), where the difference in fluorescence between OD 5 to OD 25 in the absence of farnesene was close to 5-fold. A plot of farnesene concentration versus fluorescence units across increasing cell density shows a relatively poor correlation coefficient of R²=0.650 (500ₑₓ/550ₑₘ; FIG. 2B). Thus, under spectral conditions within the yellow-gold spectrum, fluorescence can be attributable to both farnesene and biomass. These data indicate that Nile Red detection schemes which operate within the yellow-gold spectrum (excitation wavelengths of 450-500 nm and emission wavelengths of 518-550 nm) may be incompatible with applications requiring a survey of cell populations having varying cell number, for example, the high-throughput screening of libraries of WIC-producing cells. In this setting, a sample having high biomass but low WIC production may not be readily distinguishable from a sample having low biomass but high WIC production.

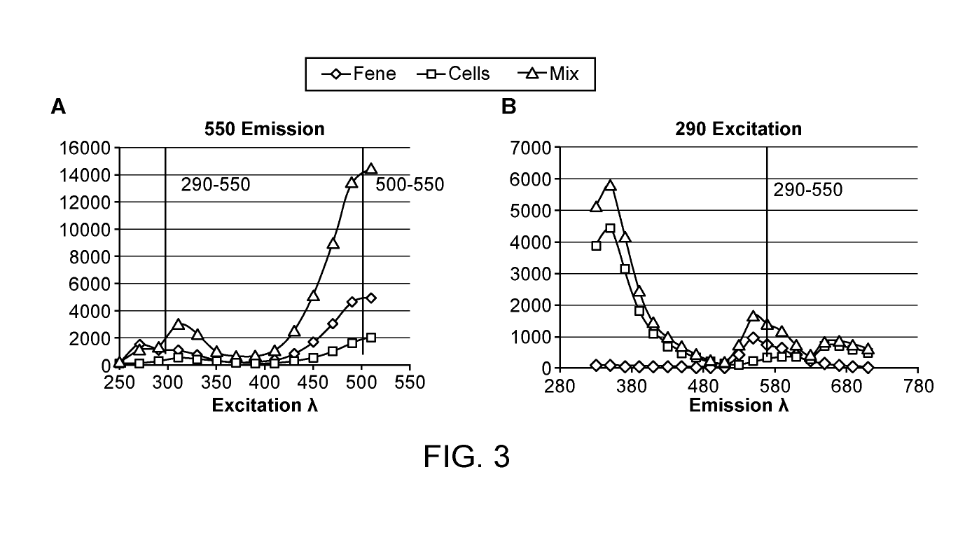

Experiments were next performed to determine whether an excitation/emission wavelength pair could be identified where the fluorescence was largely or solely attributable to farnesene, with little to no contribution by cells. In one setting, the emission wavelength was held constant at 550 nm, and an excitation spectra was generated from 250 to 520 nm (FIG. 3A). In a second setting, the excitation wavelength was held constant at 290 nm, and an emission spectra was generated from 330 to 710 nm (FIG. 3B). Three samples were tested under these spectral conditions: (1) 10 g/L farnesene, without cells; (2) naïve yeast cells of OD 25, without farnesene; and (3) 10 g/L farnesene plus naïve yeast cells of OD 25.

FIG. 3A depicts the excitation spectra at an emission wavelength of 550 nm. Consistent with previous results, detection at 500ₑₓ/550ₑₘ results in a signal of ˜2000 relative fluorescence units (RFU) for cells alone, ˜5000 RFU for farnesene alone, and ˜14000 RFU for cells plus farnesene. Thus, an artifact appears to arise at 500ₑₓ/550ₑₘ when cells are combined with farnesene, wherein fluorescence from the combination far exceeds the sum of the fluorescence from cells and farnesene, separately. By contrast, at an excitation range of 260 to 290 nm and emission at 550 nm, fluorescence from farnesene alone is no greater than farnesene plus cells, and the fluorescence from cells alone is near background levels. The excitation/emission wavelength pair of 290/550 was also observed to be favorable in view of the emission spectra at an excitation wavelength of 290 nm, as depicted in FIG. 3B. At a range of emission wavelengths from 530 to 570 nm, the fluorescence contribution from cells alone is near background levels and the farnesene only signal is near its emission peak.

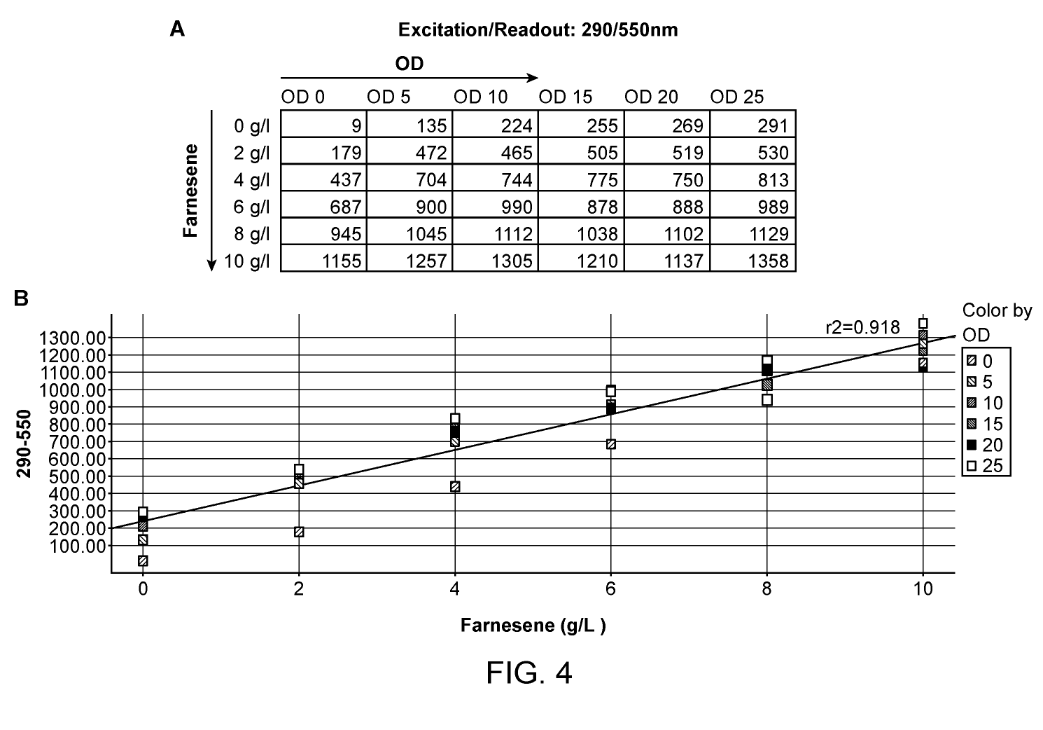

To confirm that detection of Nile Red bound to farnesene at 290ₑₓ/550ₑₘ is uninfluenced by increasing cell density, a cell/farnesene titration matrix was prepared and stained with Nile Red as described above. As shown in FIG. 4A, fluorescence increases with increasing farnesene concentration along the y-axis, but fluorescence is largely unchanged with increasing cell density along the x-axis. Furthermore, a plot of farnesene concentration versus fluorescence units across increasing cell density shows a highly improved correlation coefficient of R²=0.918 (FIG. 4B).

These results demonstrate that under select spectral conditions, e.g., an excitation wavelength of 260 to 290 nm and an emission wavelength of 530 to 570 nm, Nile Red may be used for the selective detection of farnesene, for example, farnesene recombinantly produced and secreted by a population of yeast cells, wherein fluorescence from biomass is largely eliminated. Moreover, these results provide a validation of the general methods provided herein for determining spectral conditions for a fluorescent dye that are selective for detecting dye bound to recombinantly produced water-immiscible compound.

### 7.3 Example 3

### Determination of Spectral Conditions for Specifically Detecting Biomass

The studies described in Example 2 sought to identify spectral conditions under which detection of fluorescence from Nile Red bound to farnesene is uninfluenced by fluorescence from biomass. Additional studies were carried out to identify spectral conditions under which detection of biomass via autofluorescence is uninfluenced by fluorescence from Nile Red bound to farnesene. With separate yet specific measurements of farnesene and biomass, an accurate ratio of farnesene:biomass can be obtained which may be used, for example, to stratify and rank cell populations during high-throughput Nile Red screening.

Experiments were performed to determine whether an excitation/emission wavelength pair could be identified where the fluorescence was largely or solely attributable to cell autofluorescence, with little to no contribution by Nile Red bound to farnesene. The excitation wavelength was held constant at 350 nm, and an emission spectra was generated from 430 to 750 nm. Three samples were tested under these spectral conditions: (1) 10 g/L farnesene, without cells; (2) naïve yeast cells of OD 25, without farnesene; and (3) 10 μL farnesene plus naïve yeast cells of OD 25.

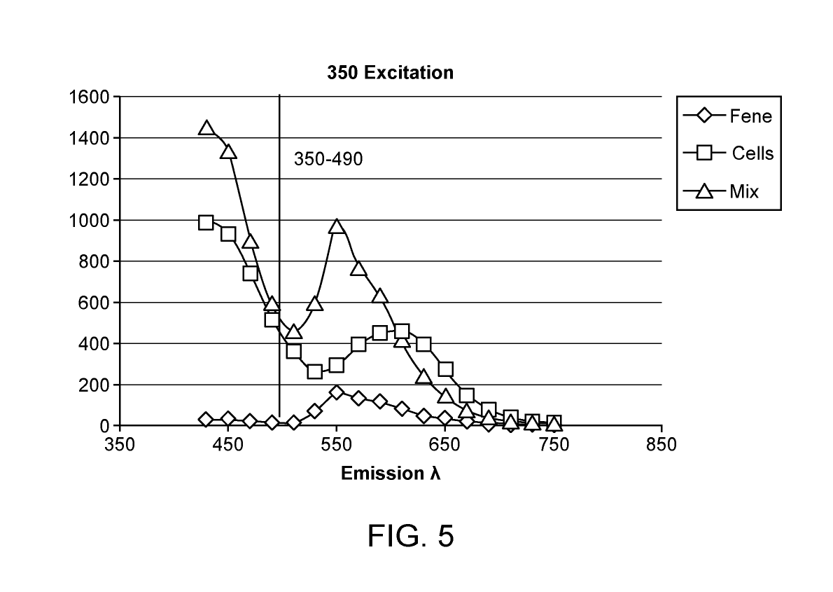

FIG. 5 depicts the emission spectra at an excitation wavelength of 350 nm. 350ₑₓ/430ₑₘ results in a signal of ˜1000 RFU for cells alone, and close to 0 RFU for farnesene alone. However, the combination of cells plus farnesene resulted in a substantial increase in fluorescence relative to cells alone (˜1450 RFU). By contrast, excitation at 350 nm and an emission range of 470 to 510 nm, fluorescence from cells plus farnesene is only slightly greater than cells alone, and the fluorescence from farnesene alone is near background levels.

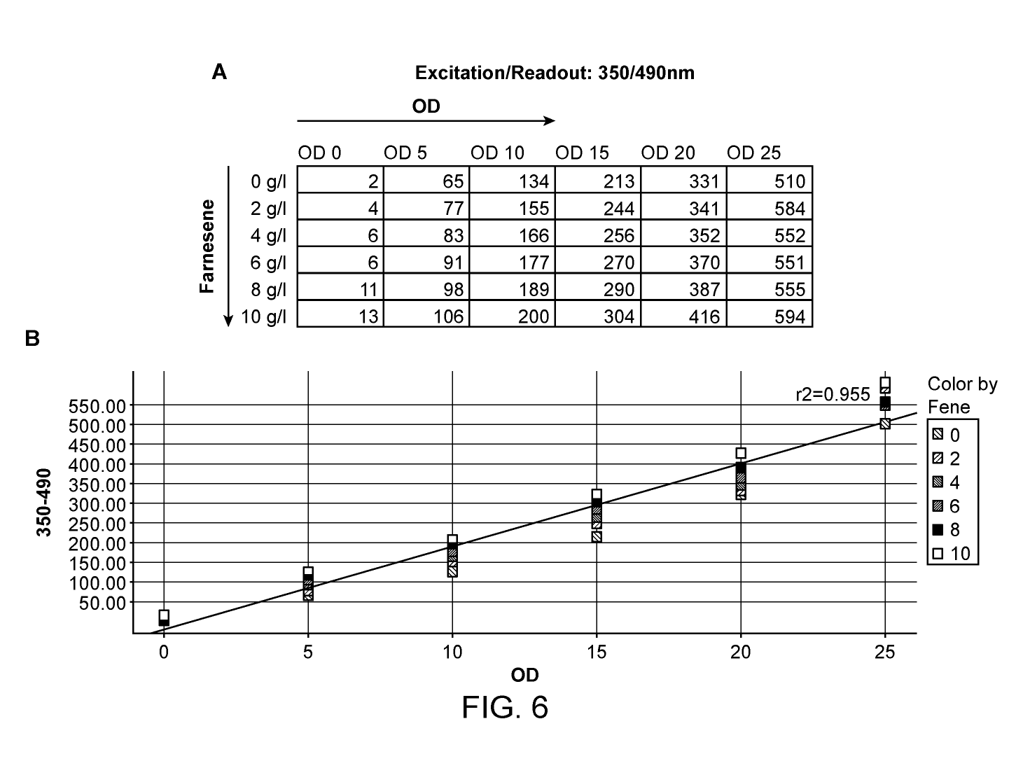

To confirm that the autofluorescence of cells 350ₑₓ/1490ₑₘ is uninfluenced by increasing farnesene, a cell/farnesene titration matrix was prepared and stained with Nile Red as described above. As shown in FIG. 6, fluorescence increases with increasing cell density along the x-axis, but fluorescence is largely unchanged with increasing farnesene concentration along the y-axis. Furthermore, a plot of cell density versus fluorescence units across increasing farnesene concentration shows a correlation coefficient of R²=0.955 (FIG. 6B). These results demonstrate that under select spectral conditions, e.g., an excitation wavelength of about 350 and an emission wavelength of 470 to 510 nm, Nile Red may be used for the selective detection of yeast cell biomass, wherein fluorescence from Nile Red bound to farnesene is largely eliminated. This method of determining an unbiased biomass reading can be extrapolated to any cell type which may be utilized for the recombinant production of WIC.

### 7.4 Example 4

### High-Throughput Screening

This example provides an exemplary method for the high-throughput Nile Red screening for farnesene production in recombinant yeast cells, prepared as described in Example 1.

Materials:

Beckman Coulter NX
M5 Spectrophotometer with stacker attachment
Black polystyrene flat bottom 96-well assay plates (Costar 3916)
INFORS Multitron II humidified shaker/incubator
(set at 33.5° C., 80% humidity, 1000 RPM)
Axygen 1.1 ml 96 well culture plates
Aeromark Breathable Membranes
Nile Red Solution (100 μg/ml in DMSO)
BSM 2% Sucrose 0.25N + crb (carbenicillin)
BSM 4% Sucrose

Preparing Pre-Culture Plates

Single colonies are picked from an agar plate into a 1.1 ml 96 well plate containing 360 μl of BSM 2% Sucrose 0.25N+crb (pre-culture media). Addition of carbenicillin to the media has been found to reduce bacterial contamination while not impacting assay performance. To maintain low coefficients of variance (CVs), all colonies are preferably picked from fresh agar plates, all treated identically. Using colonies from two sets of plates where one was stored at 4° C. for several days may lead to high CVs and uneven library performance, as quantified by the number of wells that fail to grow and perform as expected. Once inoculated with fresh colonies, pre-culture plates can be stored at 4° C. for up to 2 days with only a minor decrease in library performance.

The pre-culture plate is sealed with a breathable membrane seal, and the culture is incubated for 96 hrs at 33.5C, 80% humidity, with shaking at 1000 RPM. Breathable rayon plate seals minimize volume loss due to evaporation and allow adequate oxygen transfer to maintain an aerobic culture. When incubating multiple plates, plate position biases may be been eliminated by using a 1 cm rubber gasket to separate stacked plates. A top plate is used to cover the top of sample plates.

Dilution of Pre-Culture Plates into Production Media

14.4 μl of pre-culture media is transferred into 360 μl (1:25 dilution) of BSM 4% Sucrose (production media) contained in a 1.1 ml 96 well production plate. A dilution of pre-culture plates into production plates of 1:25 was found to be optimal for assay performance. Lower and higher dilutions were found to increase assay CVs or lengthen assay time from 48 h to 72 h or more. At a 1:25 dilution, the majority of wells are carbon exhausted after 48 h and assay CVs are maintained at normal levels.

The production plate is sealed with a breathable membrane seal, and the culture is incubated for 48 hrs at 33.5C, 80% humidity, with shaking at 1000 RPM.

Assay

Following incubation, 98 μl of production culture is mixed with 2 μL of Nile Red solution (final Nile Red concentration of 2 μg/ml) in a 96-well black polystyrene flat bottom assay plate. The plate is mixed for 30 sec. prior to loading onto the spectrophotometer. A farnesene specific read is obtained with excitation at 290 nm and emission at 550 nm, followed by a biomass specific read that is obtained with excitation at 350 nm and emission at 490 nm, and a farnesene to biomass ratio is obtained.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1-60. (canceled)

61. A liquid composition comprising:
(a) a cell recombinantly producing and secreting a water-immiscible compound;
(b) water immiscible-compound secreted from said cell;
(c) a fluorescent dye that directly binds to the secreted water-immiscible compound; and
(d) cell culture medium.

62. The composition of claim 61, wherein the cell is selected from the group consisting of a yeast cell, a bacterial cell, a mammalian cell, a fungal cell, an insect cell, and a plant cell.

63. The composition of claim 62, wherein the yeast is Saccharomyces cerevisiae.

64. The composition of claim 61, wherein the recombinantly produced water-immiscible compound is an isoprenoid.

65. The composition of claim 61, wherein the fluorescent dye is Nile Red.

66. The composition of claim 61, wherein the fluorescent dye is BODIPY 493/503 or BODIPY 505/515.

67. The composition of any claim 61, wherein the recombinantly produced water-immiscible compound is a terpene, C5 isoprenoid, C10 isoprenoid or C15 isoprenoid.

68. The composition of claim 61, wherein the recombinantly produced water-immiscible compound is farnesene.

69. The composition of claim 61, wherein the cell is a recombinant yeast cell comprising one or more heterologous nucleotide sequences encoding one or more enzymes of the mevalonate (MEV) pathway.

70. The composition of claim 69, wherein the recombinant yeast cell comprises a nucleic acid encoding farnesene synthase.

71. The composition of claim 69, wherein the recombinant yeast cell comprises a heterologous nucleotide sequence that encodes an enzyme that can convert HMG-CoA into mevalonate.

72. The composition of claim 69, wherein the recombinant yeast cell comprises a heterologous nucleotide sequence that encodes an enzyme that can convert mevalonate into mevalonate 5-phosphate.

73. The composition of claim 69, wherein the one or more heterologous nucleotide sequences encodes more than one enzyme of the mevalonate pathway.

74. The composition of claim 69, wherein the cell further comprises a heterologous nucleotide sequence encoding an enzyme that can convert isopentenyl pyrophosphate (IPP) into dimethylallyl pyrophosphate (DMAPP).

75. The composition of claim 74, wherein the cell further comprises a heterologous nucleotide sequence encoding an enzyme that can modify IPP or a polyprenyl to form an isoprenoid compound.

76. The composition of claim 75, wherein the enzyme that can modify IPP or a polyprenyl to form an isoprenoid compound is selected from the group consisting of carene synthase, geraniol synthase, linalool synthase, limonene synthase, myrcene synthase, ocimene synthase, α-pinene synthase, β-pinene synthase, γ-terpinene synthase, terpinolene synthase, amorphadiene synthase, α-farnesene synthase, β-farnesene synthase, farnesol synthase, nerolidol synthase, patchouliol synthase, nootkatone synthase, and abietadiene synthase.

77. The composition of claim 75, wherein the isoprenoid is a C₅-C₂₀ isoprenoid.

78. The composition of claim 77, wherein the isoprenoid is selected from the group consisting of abietadiene, amorphadiene, carene, α-farnesene, β-farnesene, farnesol, geraniol, geranylgeraniol, isoprene, linalool, limonene, myrcene, nerolidol, ocimene, patchoulol, β-pinene, sabinene, γ-terpinene, terpinolene, and valencene.

79. A method of detecting, in solution, farnesene produced and secreted from a ce the method comprising:
(a) contacting a solution with Nile Red, wherein the solution comprises a cell recombinantly producing and secreting farnesene; and
(b) detecting Nile Red at an excitation wavelength of about 260 to 290 nm and an emission wavelength of about 530 to 570 nm.

80. The method of claim 79, wherein the solution comprising the plurality of cells is contained in a well of a multi-well cell culture plate.

81. The method of claim 80, wherein the multi-well cell culture plate is coated with Teflon.

82. The method of claim 79, wherein the cells are cultured for a period of at least 12 hours prior to said detecting.

83. The method of claim 79, further comprising the step of shaking the multi-well cell culture plate prior to said detecting.

84. The method of claim 79, wherein the cell is selected from the group consisting of a yeast cell, a bacterial cell, a mammalian cell, a fungal cell, an insect cell, and a plant cell.

85. The method of claim 84, wherein the cell is a yeast cell.

86. The method of claim 85, wherein the yeast is Saccharomyces cerevisiae.
